(19) United States
(12) Patent Application Publication
(10) Pub. No.: US 2021/0393520 A1
(43) Pub. Date: Dec. 23, 2021

(54) TREATMENT OF GRAM-NEGATIVE FOLLICULITIS OR AN INFLAMMATION THEREOF WITH BESIFLOXACIN

(71) Applicant: VYOME THERAPEUTICS LIMITED, New Delhi (IN)

(72) Inventors: Shamik GHOSH, Ghaziabad (IN); Suresh SADHASIVAM, Salem (IN); Kalpna GARKHAL, Delhi (IN)

(73) Assignee: Vyome Therapeutics Limited, New Delhi (IN)

(21) Appl. No.: 16/967,940

(22) PCT Filed: Feb. 12, 2019
(86) PCT No.: PCT/IB2019/051119

§ 371 Date: Aug. 6, 2020

(30) Foreign Application Priority Data
Feb. 12, 2018 (IN) 201711033747

(51) Int. Cl. A61K 9/06; A61K 31/55; A61K 9/00; A61K 45/06; A61K 47/34; A61K 47/18; A61K 47/10; A61K 47/38; A61K 47/02; A61K 47/32

(52) U.S. Cl. CPC A61K 9/06; A61K 31/55; A61K 9/0014; A61K 45/06; A61K 47/32; A61K 47/183; A61K 47/10; A61K 47/38; A61K 47/02; A61K 47/34

(57) ABSTRACT

This disclosure is directed to a method of treating a patient suffering from gram-negative folliculitis or an inflammation thereof by applying a topical formulation of besifloxacin hydrochloride to the affected skin area and its surrounding.

### TECHNICAL FIELD

The present disclosure relates to a method of treating a patient suffering from gram-negative folliculitis or an inflammation associated with said gram-negative folliculitis, by applying topical formulation of Besifloxacin hydrochloride to the affected skin area and its surrounding.

### BACKGROUND OF THE DISCLOSURE

Gram negative folliculitis (GNF) is believed to be the consequence of prolong treatment with broad spectrum antibiotics. Longer duration of antibiotic regime suppresses the usual gram-positive bacterial flora of nasal and facial skin and latter facial skin is colonized by the gram-negative rod bacterial flora (Enterobacteriaceae family). A typical feature of gram-negative folliculitis includes presence of papules, pustules and sever seborrhea in perioral and perinasal area of face of the person who has undergone long time broad spectrum antibiotic treatment or of the patients who are not responding to the standard acne and rosacea treatment. Subsequent microbiological assay demonstrating predominance of gram-negative rod-shaped bacteria in facial skin and nose mucous membrane of the suspected patient confirms the diagonosis.¹ Leyden et al² observed that there are two types of gram-negative folliculitis based on their clinical symptoms and causative agent. Type I GNF is more predominant (80-90%), characterized by superficial papules and pustules present in and around the nose and mouth, caused by gram-negative, rod-shaped and lactose fermenting bacteria such as Escherichia coli, Klebsiella spp., Serratia spp. and Enterobacter spp. The Type II GNF is characterized by deep nodular and cystic infection and is caused by Proteus mirabilis.

The existing modes of treatment of GNF include antibiotics such as 3^{rd} generation cephalosporins, trimethoprim-sulfamethoxazole and Ampicillin. However, these antibiotics have high MIC values and their overuse has led to evolution of bacteria resistant to antibiotics. Thus, there exists a need for formulations based on antibiotics, which can treat GNF at relatively lower MIC and can inhibit the causal organisms, regardless of their resistance to other conventionally employed antibiotics.

### SUMMARY OF THE DISCLOSURE

The present disclosure relates to a method for treating gram-negative folliculitis in a subject, said method comprising topically administering a therapeutically effective amount of a formulation of besifloxacin at a concentration ranging from about 0.5% to about 4%. The said formulation of besifloxacin is gel, cream, lotion, foam, emulgel, ointment or spray.

In embodiments herein, the formulation, in addition to besifloxacin, comprises excipients selected from a group comprising anti-acne agent, alkalizing agent, anti-oxidant, anti-microbial agent, chelating agent, conditioning agent, dispersing agent, emollient, emulsifier, humectant, moisturizer, isotonic agent, foam stabilizer, solubilizer, thickening agent, penetration enhancer, preservative, solvent, surfactant, stabilizer, lubricant, opacifier and viscosity modifier.

In some embodiments, the alkalizing agent is selected from a group comprising sodium hydroxide and triethanolamine or a combination thereof; the anti-oxidant is selected from a group comprising butylated hydroxytoluene (BHT) and D-α-tocopherol polyethylene glycol succinate (TPGS) or a combination thereof; the anti-microbial agent is phenonip; the chelating agent is selected from a group edetate disodium and edetate disodium dihydrate or a combination thereof; the conditioning agent is cyclopentasiloxane; the dispersing agent is selected from a group comprising poloxamer 407 and poloxamer 124 or a combination thereof; the emollient is selected from a group comprising behenyl alcohol, cyclomethicone, oleyl oleate, and light liquid paraffin or a combination thereof; the emulsifier is selected from a group comprising Brij 35, cetyl alcohol, glyceryl stearate, glyceryl monostearate, laureth 4, PEG-400 stearate, polysorbate 60, steareth 2, sodium palmitate and steareth 21 or any combination thereof; the humectant is selected from a group comprising glycerine, methyl gluceth-20, and propylene glycol or a combination thereof; the moisturizer is allantoin; the isotonic agent is sodium chloride; the foam stabilizer is cocamidopropylbetaine; the solubilizer is selected from a group comprising caproyl 90, diethylene glycol monoethyl ether, N-methyl 2-pyrrolidone and polyethylene glycol 400 or any combination thereof; the thickening agent is selected from a group comprising carbomer homopolymer type C, carbomer, carbopol 980, hydroxyethyl cellulose, pemulen, sepineo P600, sodium hyaluronate, stearyl alcohol, ultrez 21 and xanthan gum or any combination thereof; the preservative is selected from a group comprising phenoxyethanol and propyl paraben; the solvent is purified water; the lubricant is PEG-7 glycerylcocoate; the opacifier is titanium dioxide; the viscosity modifier is selected from a group comprising carbopolaqua SF-1 and petrolatum; and the surfactant is selected from a group comprising sodium lauryl sulphate, sodium C14-16 olefin sulfonate, sodium lauryl ether sulphate, polyquaternium-39, ammonium lauryl sulphate (30%), disodium laureth sulfosuccinate (39%), sorbitan stearate and tween 80 or a combination thereof.

In embodiments herein, the gram-negative folliculitis is caused by gram-negative bacteria selected from a group comprising Klebsiella pneumoniae, Pseudomonas aeruginosa, Acinetobacter baumannii, Enterobacter aerogenes and Proteus mirabilis.

In further embodiments, the said gram-negative bacteria is resistant to conventional antibiotics and fluoroquinolone other than besifloxacin. These include ampicillin, amoxicillin, cefotaxime, clindamycin, tetracycline and erythromycin.

In embodiments herein, the formulation is topically administered at least once a day to up to four times a day; and wherein each administration is in an amount ranging from about 2 finger-tip unit (FTU) to about 4.5 finger-tip unit (FTU), or from about 1 gram to about 2.5 grams.

In embodiments herein, the formulation, in addition to besifloxacin, comprises a second active agent selected from a group comprising retinoid derivative, sebum inhibitor, antibiotic and anti-inflammatory agent, or any combination thereof, at a concentration ranging from about 0.001% to about 10%.

The present disclosure further relates to the said formulation for treating gram-negative folliculitis in a subject, comprising besifloxacin at a concentration ranging from about 0.5% to about 4%.

In embodiments herein, the formulation is selected from a group comprising gel, cream, lotion, foam, emulgel, ointment and spray; and in addition to the besifloxacin comprises excipients selected from a group comprising anti-acne agent, alkalizing agent, anti-oxidant, anti-microbial agent, chelating agent, conditioning agent, dispersing agent, emollient, emulsifier, humectant, moisturizer, isotonic agent, foam stabilizer, solubilizer, thickening agent, penetration enhancer, preservative, solvent, surfactant, stabilizer, lubricant, opacifier and viscosity modifier

In further embodiments, the formulation, in addition to besifloxacin, comprises a second active agent selected from a group comprising retinoid derivative, sebum inhibitor, antibiotic and anti-inflammatory agent, or any combination thereof.

The present disclosure also relates to a method for treating inflammation associated with gram-negative folliculitis in a subject, said method comprising topically administering a therapeutically effective amount of a formulation of besifloxacin at a concentration ranging from about 0.5% to about 4%.

The present disclosure also relates to a corresponding formulation for treating inflammation associated with gram-negative folliculitis in a subject, comprising besifloxacin at a concentration ranging from about 0.5% to about 4%.

### BRIEF DESCRIPTION OF THE ACCOMPANYING FIGURES

In order that the disclosure may be readily understood and put into practical effect, reference will now be made to exemplary embodiments as illustrated with reference to the accompanying figures. The figures together with a detailed description below, are incorporated in and form part of the specification, and serve to further illustrate the embodiments and explain various principles and advantages, in accordance with the present disclosure wherein:

FIG. 1 depicts the drug release profile of besifloxacin topical gel (1%) using polysulfone membrane.

FIG. 2 depicts the drug release profile of besifloxacin topical gel (2%) using polysulfone membrane.

FIG. 3 depicts the drug release profile of besifloxacin ointment (1%) using Strat-M® membrane.

FIG. 4 depicts the drug release profile of besifloxacin emulgel (1%) using Strat-M® membrane.

FIG. 5 depicts the besifloxacin drug retention from besifloxacin cream on pig ear skin.

FIG. 6 depicts the besifloxacin drug retention from besifloxacin gel on pig ear skin.

FIG. 7 depicts the besifloxacin drug retention from besifloxacin cream on human skin.

FIG. 8 depicts the besifloxacin drug retention from besifloxacin gel on human skin.

### DETAILED DESCRIPTION

In view of the drawbacks associated, and to remedy the need created by the art available in the field of medicine, it is an objective of the disclosure to provide a new topical composition and a method for treating gram negative folliculitis, hot tub folliculitis or any other inflammatory disease of pilo-sebaceous follicles of skin, which is caused as a result of a new infection or de novo proliferation of commensal gram-negative bacteria.

The present disclosure thus provides a method of effectively treating gram-negative folliculitis or an inflammation associated with gram-negative folliculitis. The said treatment is carried out by topically administering a formulation that comprises besifloxacin at a specific concentration.

Accordingly, the present disclosure relates to a method for treating gram-negative folliculitis in a subject, said method comprising topically administering a therapeutically effective amount of a formulation of besifloxacin at a concentration ranging from about 0.5% to about 4%.

Similarly, the present disclosure relates to a method for treating inflammation associated with gram-negative folliculitis in a subject, said method comprising topically administering a therapeutically effective amount of a formulation of besifloxacin at a concentration ranging from about 0.5% to about 4%.

The besifloxacin employed in the present disclosure for treatment of gram-negative folliculitis or inflammation thereof is in different formulations including, but not limited to gel, cream, lotion, foam, emulgel, ointment and spray.

These formulations of besifloxacin are either aqueous or non-aqueous formulations. Accordingly, in embodiments of the present disclosure, when the formulation is non-aqueous, it is pH independent, whereas, when the formulation is an aqueous formulation, the pH of such formulation ranges from about 5 to about 8.

The said formulations of besifloxacin employed in the present disclosure for treatment of gram-negative folliculitis or an inflammation thereof comprises one or more components beyond besifloxacin itself. These components improve the activity and bioavailability of besifloxacin, and include anti-acne agent, alkalizing agent, anti-oxidant, anti-microbial agent, chelating agent, conditioning agent, dispersing agent, emollient, emulsifier, humectant, moisturizer, isotonic agent, foam stabilizer, solubilizer, thickening agent, penetration enhancer, preservative, solvent, surfactant, stabilizer, lubricant, opacifier and viscosity modifier.

In non-limiting embodiments of the present disclosure, the alkalizing agent is selected from a group comprising sodium hydroxide and triethanolamine or a combination thereof. When employed in a formulation of the present disclosure, the sodium hydroxide is at a concentration ranging from about 0.04% to about 1.2%, and the triethanolamine is at a concentration of about 1%.

In other non-limiting embodiments of the present disclosure, the anti-oxidant is selected from a group comprising butylated hydroxytoluene (BHT) and D-α-Tocopherol polyethylene glycol succinate (TPGS) or a combination thereof. When employed in a formulation of the present disclosure, the butylated hydroxytoluene (BHT) is at a concentration of about 0.1%, and the D-α-Tocopherol polyethylene glycol succinate (TPGS) is at a concentration ranging from about 3% to about 5%.

In further non-limiting embodiments of the present disclosure, the anti-microbial agent is phenonip. When employed in a formulation of the present disclosure, the phenonip is at a concentration ranging from about 0.3% to about 0.4%.

In further non-limiting embodiments of the present disclosure, the chelating agent is selected from a group edetate disodium and edetate disodium dihydrate or a combination thereof. When employed in a formulation of the present disclosure, the edetate disodium or the edetate disodium dihydrate is at a concentration of about 0.1%.

In further non-limiting embodiments of the present disclosure, the conditioning agent is cyclopentasiloxane. When employed in a formulation of the present disclosure, the cyclopentasiloxane is at a concentration of about 5%.

In further non-limiting embodiments of the present disclosure, the dispersing agent is selected from a group comprising poloxamer 407 and poloxamer 124 or a combination thereof. When employed in a formulation of the present disclosure, the poloxamer 407 or the poloxamer 124 is at a concentration ranging from about 0.5% to about 1%.

In further non-limiting embodiments of the present disclosure, the emollient is selected from a group comprising behenyl alcohol, cyclomethicone, oleyl oleate, and light liquid paraffin or a combination thereof. When employed in a formulation of the present disclosure, the behenyl alcohol is at a concentration ranging from about 1% to about 1.5%, the cyclomethicone is at a concentration ranging from about 1% to about 6%, the oleyl oleate is at a concentration of about 0.5%, and the light liquid paraffin is at a concentration ranging from about 2% to about 7%.

In further non-limiting embodiments of the present disclosure, the emulsifier is selected from a group comprising Brij 35, cetyl alcohol, glyceryl stearate, glyceryl monostearate, laureth 4, PEG-400 stearate, polysorbate 60, steareth 2, sodium palmitate and steareth 21 or any combination thereof. When employed in a formulation of the present disclosure, the Brij 35 is at a concentration of about 5.1%, the cetyl alcohol is at a concentration ranging from about 1% to about 2%, the glyceryl stearate or the glyceryl monostearate is at a concentration ranging from about 1.5% to about 3%, the laureth 4 is at a concentration of about 4%, the PEG-400 stearate is at a concentration ranging from about 2% to about 10%, the polysorbate 60 is at a concentration ranging from about 2% to about 4%, the sodium palmitate is at a concentration of about 94.2% and the steareth 2 or the steareth 21 is at a concentration ranging from about 2% to about 3%.

In further non-limiting embodiments of the present disclosure, the humectant is selected from a group comprising glycerin, methyl gluceth-20 and propylene glycol or a combination thereof. When employed in a formulation of the present disclosure, the glycerin is at a concentration ranging from about 1% to about 10%, methyl gluceth-20 is at a concentration ranging of about 0.3% to about 2.5% and the propylene glycol is at a concentration ranging from about 1% to about 22%.

In further non-limiting embodiments of the present disclosure, the moisturizer is allantoin. When employed in a formulation of the present disclosure, the allantoin is at a concentration of about 0.2%.

In further non-limiting embodiments of the present disclosure, the isotonic agent is sodium chloride. When employed in a formulation of the present disclosure, the sodium chloride is at a concentration of about 0.9%.

In further non-limiting embodiments of the present disclosure, the foam stabilizer is cocamidopropylbetaine. When employed in a formulation of the present disclosure, the cocamidopropylbetaine is at a concentration of about 0.5%.

In further non-limiting embodiments of the present disclosure, the solubilizer is selected from a group comprising caproyl 90, diethylene glycol monoethyl ether, N-methyl 2-pyrrolidone and polyethylene glycol 400 or any combination thereof. When employed in a formulation of the present disclosure, the caproyl 90 is at a concentration ranging from about 4% to about 5%, the diethylene glycol monoethyl ether is at a concentration ranging from about 1% to about 16%, the N-methyl 2-pyrrolidone is at a concentration of about 3%, and the polyethylene glycol 400 is at a concentration ranging from about 0.1% to about 8%.

In further non-limiting embodiments of the present disclosure, the thickening agent is selected from a group comprising carbomer homopolymer type C, carbomer, carbopol 980, hydroxyethyl cellulose, pemulen, sepineo P600, sodium hyaluronate, stearyl alcohol, ultrez 21 and xanthan gum or any combination thereof. When employed in a formulation of the present disclosure, the carbomer homopolymer type C is at a concentration ranging from about 0.3% to about 0.65%, the carbomer or the carbopol 980 is at a concentration ranging from about 0.1% to about 25%, the hydroxyethyl cellulose is at a concentration ranging from about 0.17% to about 1.75%, the pemulen is at a concentration ranging from about 1% to about 40%, the sepineo P600 is at a concentration ranging from about 4% to about 5%, the sodium hyaluronate is at a concentration ranging from about 0.1% to about 0.5%, the stearyl alcohol is at a concentration ranging from about 1% to about 2%, the ultrez 21 is at a concentration ranging from about 5% to about 20%, and the xanthan gum is at a concentration ranging from about 0.5% to about 0.6%.

In further non-limiting embodiments of the present disclosure, the preservative is phenoxyethanol or propyl paraben. When employed in a formulation of the present disclosure, the phenoxyethanol is at a concentration ranging from about 0.3% to about 0.7%, and the propyl paraben is at a concentration of about 0.03%.

In further non-limiting embodiments of the present disclosure, the surfactant is selected from a group comprising sodium lauryl sulphate, sodium C14-16 olefin sulfonate, sodium lauryl ether sulphate, polyquaternium-39, ammonium lauryl sulphate (30%), disodium laureth sulfosuccinate (39%), sorbitan stearate and tween 80 or a combination thereof. When employed in a formulation of the present disclosure, the sodium lauryl sulphate is at a concentration of about 5%, the sodium C14-16 olefin sulfonate is at a concentration of about 35%, the sodium lauryl ether sulphate is at a concentration of about 2%, the polyquaternium-39 is at a concentration of about 1%, the ammonium lauryl sulphate (30%) is at a concentration of about 30%, the disodium laureth sulfosuccinate (39%) is at a concentration of about 2%, the sorbitan stearate is at a concentration of about 1.4% and the tween 80 is at a concentration of about 8%.

In further non-limiting embodiments of the present disclosure, the lubricant is PEG-7 glycerylcocoate. When employed in a formulation of the present disclosure, the PEG-7 glycerylcocoate is at a concentration of about 1%.

In further non-limiting embodiments of the present disclosure, the opacifier is titanium dioxide. When employed in a formulation of the present disclosure, the titanium dioxide is at a concentration of about 0.5%.

In further non-limiting embodiments of the present disclosure, the viscosity modifier is selected from a group comprising carbopolaqua SF-1 and petrolatum. When employed in a formulation of the present disclosure, the carbopolaqua SF-1 is at a concentration ranging from about 1% to about 6%, and the petrolatum is at a concentration of about 1%.

In further non-limiting embodiments of the present disclosure, the solvent employed for preparing the formulations is purified water.

While the formulations of the present disclosure primarily comprise besifloxacin, they may also comprise of a second active agent. These active agents in combination with besifloxacin help in enhancing the treatment of the gram-negative folliculitis or an inflammation thereof.

Accordingly, in embodiments of the present disclosure, the formulation in addition to besifloxacin, comprises a second active agent selected from a group comprising retinoid derivative, sebum inhibitor, antibiotic and anti-inflammatory agent, or any combination thereof. This second active agent is at a concentration ranging from about 0.001% to about 10%.

In non-limiting embodiments of the present disclosure, the retinoid derivative is selected from a group comprising isotretinoin, tretinoin, tazoretene and adapalene or any combination thereof. When employed in a formulation, the retinoid derivative is at a concentration ranging from about 0.025% to about 0.7%. In an exemplary embodiment, the isotretinoin is at a concentration ranging from about 0.025% to about 0.7%, the tretinoin is at a concentration ranging from about 0.025% to about 0.1%, and the adapalene is at a concentration of about 0.1%.

In non-limiting embodiments of the present disclosure, the sebum inhibitor is selected from a group comprising acetyle coenzyme A carboxylase inhibitor and olumacostat glasaretil or a combination thereof. When employed in a formulation, the sebum inhibitor is at a concentration ranging from about 0.1% to about 10%.

In non-limiting embodiments of the present disclosure, the antibiotic is selected from a group comprising sulphadiazine and metronidazole or a combination thereof. When employed in a formulation, the antibiotic is at a concentration ranging from about 0.1% to about 10%.

In non-limiting embodiments of the present disclosure, the anti-inflammatory agent is selected from a group comprising nonsteroidal anti-inflammatory drug, cox-2 inhibitor, JAK inhibitor, PDE4 inhibitor, anti-leukotriene, aspirin, ibuprofen, naproxen, diclofenac, and nimesulide, or any combination thereof. Further, the COX-2 inhibitor is selected from a group comprising Celecoxib, rofecoxib, and valdecoxib, or any combination thereof; the JAK inhibitor is selected from a group comprising tofacitinib, ruxolitinib, baricitinib, oclacitinib and baricitinib, or any combination thereof; the PDE4 inhibitor is selected from a group comprising apremilast, roflumilast, cilomilast, ibudilast, piclamilast and crisaborole, or any combination thereof, and the anti-leukotriene is a LTB4 pathway drug selected from a group comprising etalocib, amelubant, moxilubant, ubenimex, tosedostat and acebilustat.

When employed in a formulation, the anti-inflammatory agent is at a concentration ranging from about 0.1% to about 10%.

The besifloxacin based formulations of the present disclosure treat the gram-negative folliculitis or an inflammation thereof by inhibiting the causal organisms thereof. In non-limiting embodiments, the gram-negative folliculitis is caused by gram-negative bacteria selected from a group comprising Escherichia coli, Klebsiella spp., Pseudomonas spp., Serratia spp., Acinetobacter spp., Enterobacter spp. and Proteus spp.

In exemplary embodiments, the gram-negative folliculitis is caused by gram-negative bacteria selected from a group comprising Klebsiella pneumoniae, Pseudomonas aeruginosa, Acinetobacter baumannii, Enterobacter aerogenes and Proteus mirabilis.

Accordingly, the besifloxacin based formulations of the present disclosure inhibit the gram-negative bacteria causing the folliculitis, and the minimum inhibitory concentration (MIC) of the besifloxacin against said gram-negative bacteria is lower than the MIC of conventional antibiotics such as cefotaxime and ampicillin. Further, the besifloxacin based formulations of the present disclosure are also able to treat the gram-negative folliculitis or an inflammation, caused by a gram-negative bacteria which is resistant to conventional antibiotics and fluoroquinolone other than besifloxacin.

In embodiments of the present disclosure, the besifloxacin also inhibits gram-positive bacteria associated with acne vulgaris selected from a group comprising Propionibacterium acnes, Staphylococcus epidermidis, Staphylococcus haemolyticus, Bacillus megaterium, Dermabacter hominis, Kocuria spp., Microbacterium spp., and Blastococcus spp.

As mentioned previously, the besifloxacin based formulations of the present disclosure are administered or applied topically for curing the gram-negative folliculitis. In non-limiting embodiments of the present disclosure, the said administration or application is carried out at least once a day to up to four times a day, to a subject suffering from gram-negative folliculitis. in need thereof. In embodiments herein, the subject is a human being suffering from said gram-negative folliculitis.

The amount of the besifloxacin based formulations of the present disclosure that must be topically administered or applied each time, ranges from about 2 finger-tip unit (FTU) to about 4.5 finger-tip unit (FTU). In other words, and in terms of grams, the amount of the besifloxacin based formulations of the present disclosure that must be topically administered or applied each time, ranges from about 1 gram to about 2.5 grams.

Thus, the topical administration of the besifloxacin based formulation is carried out at least once a day to up to four times a day; and wherein each administration is in an amount ranging from about 2 finger-tip unit (FTU) to about 4.5 finger-tip unit (FTU), or from about 1 gram to about 2.5 grams.

Thus, in exemplary embodiments, the amount of the besifloxacin based formulations of the present disclosure that must be topically administered or applied, ranges from about 2 FTU per day to about 18 FTU per day. In other words, the total of amount of besifloxacin based formulations of the present disclosure that must be topically administered or applied, ranges from about 1 gram per day to about 10 grams per day.

While the present disclosure provides formulations such as gel, cream, lotion, foam, emulgel, ointment and spray, to treat the gram-negative folliculitis or an inflammation thereof, it also provides corresponding processes to obtain the formulations. Since the formulations comprise of various components other than besifloxacin in different concentrations, different formulations may require slightly different processes to prepare them. However, the processes provided by this disclosure are not absolute, as a person skilled in the art will understand that once the components of a formulation are listed, how to best combine them for an effective formulation. Thus, in that regard, the importance of the present disclosure lies on providing the formulations and the components that make them.

Accordingly, the present disclosure provides a gel formulation of besifloxacin for treatment of gram-negative folliculitis or an inflammation thereof. Such a formulation at least comprises components selected from a group comprising besifloxacin, humectant, chelating agent, thickening agent, preservative, solubilizer and alkalizing agent.

In an exemplary embodiment, such a formulation comprises glycerin, besifloxacin HCl, purified water, edetate disodium, hydroxyethyl cellulose, sodium hyaluronate, carbomer 980, phenoxyethanol, polyethylene glycol 400, diethylene glycol monoethyl ether, and sod. hydroxide (18% w/w).

In another exemplary embodiment, such a formulation comprises besifloxacin.HCl, edetate disodium dihydrate, carbomer homopolymer type c, diethylene glycol monoethyl ether, polyethylene glycol 400, hydroxyethyl cellulose, sepineo p600, sodium hyaluronate, phenoxyethanol, glycerin, sodium hydroxide solution and purified water.

The gel formulations herein, in addition to above components, may further comprise moisturizer, dispersing agent, surfactant, retinoid derivative and isotonic agent.

In an exemplary embodiment, such a formulation comprises purified water, edetate disodium, allantoin, hydroxyethyl cellulose, carbomer 980, purified water, sodium hyaluronate, poloxamer 407, phenoxyethanol, sodium hydroxide (18% w/v), glycerin, besifloxacin HCl equivalent to besifloxacin, purified water, sodium hydroxide solution, polyethylene glycol 400, diethylene glycol monoethyl ether and sodium hydroxide (18% w/v).

In another exemplary embodiment, such a formulation comprises purified water, allantoin, carbomer 980, sodium hyaluronate, poloxamer 407, besifloxacin HCl, glycerin, polyethylene glycol 400, edetate disodium, phenoxyethanol, triethanolamine and diethylene glycol monoethyl ether.

In yet another exemplary embodiment, such a formulation comprises besifloxacin.HCl equivalent to besifloxacin, sodium lauryl sulphate (30%), tween 80, diethylene glycol monoethyl ether, propylene glycol, disodium edetate, sodium hydroxide (10%), carbomer 980 (3%), water, phenoxyethanol and triethanolamine.

In yet another exemplary embodiment, such a formulation comprises glycerin, besifloxacin HCl, purified water, edetate disodium, hydroxyethyl cellulose, sodium hyaluronate, carbomer 980, phenoxyethanol, isotretinoin, tretinoin, poloxamer 407, polyethylene glycol 400, diethylene glycol monoethyl ether and sod. hydroxide (18% w/w).

In yet another exemplary embodiment, such a formulation comprises glycerin, besifloxacin HCl, purified water, edetate disodium, hydroxyethyl cellulose, sodium hyaluronate, carbomer 980, phenoxyethanol, polyethylene glycol 400, diethylene glycol monoethyl ether, sodium chloride and sod. hydroxide solution.

Similarly, the present disclosure provides a cream formulation of besifloxacin for treatment of gram-negative folliculitis or an inflammation thereof. Such a formulation at least comprises components selected from a group comprising besifloxacin, thickening agent, emulsifier, humectant, alkalizing agent and anti-oxidants. In addition, such formulations can also comprise further components such as conditioning agent, anti-microbial agent, emollient, retinoid derivative, dispersing agent, solubilizer and preservative.

In an exemplary embodiment, such a formulation comprises pemulen TR1, cyclopentasiloxane, cetyl alcohol, stearyl alcohol, steareth 2, besifloxacin.HCl equivalent to besifloxacin, propylene glycol, steareth 21, water, sodium hydroxide (10%), BHT and phenonip.

In another exemplary embodiment, such a formulation comprises cyclomethicone, cetyl alcohol, stearyl alcohol, steareth 2, steareth 21, light liquid paraffin, besifloxacin HCl equivalent to besifloxacin, sodium hydroxide solution (10%), water, glycerol, carbopol 980 (2%), sodium hydroxide solution (10%), water, isotretinoin, tretinoin, poloxamer 407, water, PEG 400, butylated hydroxy toluene and phenoxyethanol.

The present disclosure also provides a lotion formulation of besifloxacin for treatment of gram-negative folliculitis or an inflammation thereof. Such a formulation at least comprises components selected from a group comprising besifloxacin, thickening agent, humectant, alkalizing agent and preservative. In addition, such formulations can also comprise further components such as anti-oxidant, emollient, emulsifier, chelating agent, solubilizer and isotonic agent.

In an exemplary embodiment, such a formulation comprises PEG-400 stearate, glyceryl stearate, light liquid paraffin, behenyl alcohol, glycerol, propylene glycol, besifloxacin HCl equivalent to besifloxacin, water, sodium hydroxide, carbopol 980, xanthan gum, sepineo P600, water, BHT and phenoxyethanol.

In another exemplary embodiment, such a formulation comprises glycerin, besifloxacin HCl, purified water, edetate disodium, hydroxyethyl cellulose, sodium hyaluronate, carbomer 980, phenoxyethanol, polyethylene glycol 400, diethylene glycol monoethyl ether, sodium chloride and sod. hydroxide solution.

In yet another exemplary embodiment, such a formulation comprises glycerin, besifloxacin HCl, purified water, edetate disodium, hydroxyethyl cellulose, sodium hyaluronate, carbomer 980, phenoxyethanol, polyethylene glycol 400, diethylene glycol monoethyl ether, sodium chloride and sod. hydroxide solution.

The present disclosure further provides a form formulation of besifloxacin for treatment of gram-negative folliculitis or an inflammation thereof. Such a formulation comprises components selected from a group comprising besifloxacin, thickening agent, emulsifier, isotonic agent, humectant, alkalizing agent, anti-oxidant, retinoid derivative and preservative.

In an exemplary embodiment, such a formulation comprises cetyl alcohol, glyceryl monostearate, cocamidopropylbetaine, steareth 2, steareth 21, polysorbate 60, glycerol, propylene glycol, besifloxacin.HCl equivalent to besifloxacin, water, sodium hydroxide, carbomer 980, sodium hydroxide, water, isotretinoin, tretinoin, BHT and phenoxyethanol.

The present disclosure further provides a spray formulation of besifloxacin for treatment of gram-negative folliculitis or an inflammation thereof. Such a formulation comprises components selected from a group comprising besifloxacin, humectant, thickening agent, preservative and alkalizing agent.

In an exemplary embodiment, such a formulation comprises glycerin, besifloxacin HCl, sodium carboxy methyl cellulose, phenoxyethanol, sodium hydroxide and purified water.

Similarly, the present disclosure also provides an emulgel formulation of besifloxacin for treatment of gram-negative folliculitis or an inflammation thereof. Such a formulation at least comprises components selected from a group comprising besifloxacin, emulsifier, anti-oxidant, humectant, thickening agent, solubilizer, alkalizing agent and dispersing agent.

In an exemplary embodiment, such a formulation comprises caproyl 90, cetyl alcohol, steareth 2, laureth 4, BRIJ 35, besifloxacin.HCl equivalent to besifloxacin, steareth 21, D-α-tocopherol polyethylene glycol 1000 succinate (tpgs), poloxamer 407, poloxamer 124, propylene glycol, purified water, propylene glycol, purified water, ultrez 21 (0.5% w/w) and triethanolamine.

The emulgel formulations herein, in addition to above components, may further comprise preservative, retinoid derivative and emollient.

In an exemplary embodiment, such a formulation comprises besifloxacin.HCl equivalent to besifloxacin, caproyl 90, cetyl alcohol, steareth 2, steareth 21, D-α-tocopherol polyethylene glycol 1000 succinate (tpgs), poloxamer 407, poloxamer 124, propylene glycol, purified water, ultrez 21 (2% w/w), isotretinoin, tretinoin, PEG 500, water, triethanolamine and phenoxyethanol.

In another exemplary embodiment, such a formulation comprises besifloxacin.HCl equivalent to besifloxacin, steareth 21, D-α-tocopherol polyethylene glycol 1000 succinate (tpgs), poloxamer 407, poloxamer 124, propylene glycol, purified water, caproyl 90, cetyl alcohol, steareth 2, cyclomethicone, ultrez 21 (1% w/w) and triethanolamine.

In another embodiment herein, a formulation of the present disclosure comprises Water, Carbopol 940, Allantoin, Besifloxacin HCl, (equivalent to besifloxacin), Adapalene, Triethanolamine, Glycerol, Propylene Glycol, PEG 400, Poloxamer 407, Disodium EDTA and Phenoxyethanol.

In yet another embodiment herein, a formulation of the present disclosure comprises Water, Sodium hydroxide (18% aq.), PEG 1450, Methyl Gluceth-20, Glycerin, Besifloxacin, Adapalene, Isopropyl alcohol, diethylene glycol monethyl ether, Propylene glycol, N-methyl 2-pyrrolidone, Sodium hydroxide, Phenoxyethanol and Fragrance

In still another embodiment herein, a formulation of the present disclosure comprises Water, Carbopol aqua SF-1, Sodium C14-16 Olefin Sulfonate, Sodium lauryl ether sulphate, Sodium hydroxide (18% aq.), Cocamidopropylbetaine (30%), Disodium EDTA, Glycerin, Besifloxacin, Adapalene, N-methyl 2-pyrrolidone, PEG-7 glycerylcocoate and Citric Acid (50%)

In still another embodiment herein, a formulation of the present disclosure comprises Sodium palmitate, Sodium lauryl ether sulphate, Polyquaternium-39, Methyl Gluceth-20, Titanium dioxide, Besifloxacin, Adapalene, Oleyl oleate and BHT (Butylated Hydorxy Toluene)

In still another embodiment herein, a formulation of the present disclosure comprises Water, Disodium EDTA, Carbopolaqua SF-1, Ammonium lauryl sulphate (30%), Propylene glycol, Besifloxacin, Adapalene, N-methyl 2-pyrrolidone, Ethanol, Propyl paraben, Methyl gluceth-10, Disodium laureth sulfosuccinate (39%), Fragrance and Triethanolamine

In still another embodiment herein, a formulation of the present disclosure comprises Water, Disodium EDTA, Carbopol aqua SF-1, Petrolatum, Cyclomethicone, Sorbitan stearate, Polysorbate 60, Methyl gluceth-20, Cetyl alcohol, Tocopheryl acetate, Besifloxacin, Adapalene, N-methyl 2-pyrrolidone, Propylene glycol, Glycerin, Ethanol, Phenoxyethanol and Sodium hydroxide

In still another embodiment herein, a formulation of the present disclosure comprises Besifloxacin.HCl Equivalent to Besifloxacin, Adapalene, Allantoin, Diethylene glycol monoethyl ether, Edetate disodium dihydrate, Glycerin, Hyaluronate Sodium, Hydroxy ethylcellulose, Phenoxyethanol, Poloxamer, Polyethylene glycol 400, and Purified water.

In still another embodiment herein, a formulation of the present disclosure comprises Adapalene, Besifloxacin.HCl equivalent to besifloxacin, Allantoin, Citric acid solution, Diethylene glycol monoethyl ether, Edetate disodium dehydrate, Glycerin, Hyaluronate Sodium, Hydroxy ethyl cellulose, Phenoxyethanol, Poloxamer 407, Polyethylene glycol 400, Sodium Hydroxide Solution and Purified Water.

In still another embodiment herein, a formulation of the present disclosure comprises Besifloxacin.HCl equivalent to besifloxacin, Adapalene, Allantoin, Carbomer homopolymer type C, Diethylene glycol monoethyl ether, Edetate disodium dehydrate, Glycerin, Hyaluronate sodium, Phenoxyethanol, Poloxamer 407, Polyethylene glycol 400, Sodium hydroxide solution and Purified water.

As a person skilled in the art understands, while the concentrations of the components employed in different formulations differ slightly, the overall concentration of every component employed in formulations of the present disclosure is governed by the ranges provided herein. Accordingly, the sodium hydroxide is at a concentration ranging from about 0.04% to about 1.2%, the triethanolamine is at a concentration of about 1%, the butylated hydroxytoluene (BHT) is at a concentration of about 0.1%, the D-α-Tocopherol polyethylene glycol succinate (TPGS) is at a concentration ranging from about 3% to about 5%, the phenonip is at a concentration ranging from about 0.3% to about 0.4%, the edetate disodium or the edetate disodium dihydrate is at a concentration of about 0.1%, the cyclopentasiloxane is at a concentration of about 5%, the poloxamer 407 or the poloxamer 124 is at a concentration ranging from about 0.5% to about 1%, the behenyl alcohol is at a concentration ranging from about 1% to about 1.5%, the cyclomethicone is at a concentration ranging from about 1% to about 6%, the oleyl oleate is at a concentration of about 0.5%, the light liquid paraffin is at a concentration ranging from about 2% to about 7%, the Brij 35 is at a concentration of about 5.1%, the cetyl alcohol is at a concentration ranging from about 1% to about 2%, the glyceryl stearate or the glyceryl monostearate is at a concentration ranging from about 1.5% to about 3%, the laureth 4 is at a concentration of about 4%, the PEG-400 stearate is at a concentration ranging from about 2% to about 10%, the polysorbate 60 is at a concentration ranging from about 2% to about 4%, the sodium palmitate is at a concentration of about 94.2%, the steareth 2 or the steareth 21 is at a concentration ranging from about 2% to about 3%, the glycerin is at a concentration ranging from about 1% to about 10%, Methyl Gluceth-20 is at a concentration ranging of about 0.3% to about 2.5%, the propylene glycol is at a concentration ranging from about 1% to about 22%, the allantoin is at a concentration of about 0.2%, the sodium chloride is at a concentration of about 0.9%, the cocamidopropylbetaine is at a concentration of about 0.5%, the caproyl 90 is at a concentration ranging from about 4% to about 5%, the diethylene glycol monoethyl ether is at a concentration ranging from about 1% to about 16%, the N-methyl 2-pyrrolidone is at a concentration of about 3%, the polyethylene glycol 400 is at a concentration ranging from about 0.1% to about 8%, the carbomer homopolymer type C is at a concentration ranging from about 0.3% to about 0.65%, the carbomer or the carbopol 980 is at a concentration ranging from about 0.1% to about 25%, the hydroxyethyl cellulose is at a concentration ranging from about 0.17% to about 1.75%, the pemulen is at a concentration ranging from about 1% to about 40%, the sepineo P600 is at a concentration ranging from about 4% to about 5%, the sodium hyaluronate is at a concentration ranging from about 0.1% to about 0.5%, the stearyl alcohol is at a concentration ranging from about 1% to about 2%, the ultrez 21 is at a concentration ranging from about 5% to about 20%, the xanthan gum is at a concentration ranging from about 0.5% to about 0.6%, the phenoxyethanol is at a concentration of about 0.5%, the propyl paraben is at a concentration of about 0.03%, glycerylcocoate is at a concentration of about 1%, the titanium dioxide is at a concentration of about 0.5%, the carbopolaqua SF-1 is at a concentration ranging from about 1% to about 6%, the petrolatum is at a concentration of about 1%, the sodium C14-16 olefin sulfonate is at a concentration of about 35%, the sodium lauryl ether sulphate is at a concentration of about 2%, the polyquaternium-39 is at a concentration of about 1%, the ammonium lauryl sulphate (30%) is at a concentration of about 30%, the disodium laureth sulfosuccinate (39%) is at a concentration of about 2%, the sorbitan stearate is at a concentration of about 1.4%, the isotretinoin is at a concentration ranging from about 0.025% to about 0.7%, the tretinoin is at a concentration ranging from about 0.025% to about 0.1%, sodium lauryl sulphate is at a concentration of about 5%, and the tween 80 is at a concentration of about 8%.

In a preferred embodiment, the present disclosure provides a besifloxacin formulation for treatment of gram-negative folliculitis or inflammation thereof, comprising: about 0.5 to about 4 (% w/w) besifloxacin.HCl (Equivalent to besifloxacin); about 2 to about 7 (% w/w) diethylene glycol monoethyl ether; about 0.1 (% w/w) Edetate disodium dihydrate (EDTA); about 2 to about 10 (% w/w) glycerin; about 0.9 to about 1.75 (% w/w) hydroxyethyl cellulose; 0 to about 0.8 (% w/w) carbomer; about 0.3 to about 0.7 (% w/w) phenoxyethanol; about 2 to about 7 (% w/w) polyethylene glycol 400; 0 to about 0.5 (% w/w) sodium hyaluronate; sodium hydroxide; and purified water.

In another preferred embodiment, the present disclosure provides a besifloxacin formulation for treatment of gram-negative folliculitis or inflammation thereof, comprising: about 1 to about 4 (% w/w) besifloxacin.HCl (Equivalent to Besifloxacin); about 5 (% w/w) diethylene glycol mono ethyl ether; about 0.1 (% w/w) Edetate disodium dihydrate (EDTA); about 5 (% w/w) glycerin; about 0.5 to about 1.5 (% w/w) hydroxyethyl cellulose; about 0.3 to about 1.2 (% w/w) carbomer; about 0.7 (% w/w) phenoxyethanol; about 5 (% w/w) polyethylene glycol 400; 0 to about 1 (% w/w) sodium hyaluronate; sodium hydroxide; and purified water.

As mentioned previously, each of these formulations comprise besifloxacin at a concentration ranging from about 0.5% to about 4%, thereby making the formulation effective against microorganisms that cause gram-negative folliculitis. The MIC of besifloxacin against such microorganism ranges from about 0.125 μg/ml to about 16 μg/ml. This MIC is better by at least 50% when compared with other conventional antibiotic such as cefotaxime, ampicillin, clindamycin, tetracycline and erythromycin.

Further, the formulations of the present disclosure also showcase enhanced drug release properties. Drug release is an important property of a therapeutic agent and is a prerequisite for its absorption and penetration of drug to the site of action on the skin layer. Demonstration of drug release from a formulation attests to its availability on the hair follicles and upper layer of skin where pathogen responsible for disease resides, thus resulting in its activity against the target disease/condition. The data from the present disclosure suggests that amount of drug released and retained onto the skin from the besifloxacin based formulations of the present disclosure, is sufficient to exert its therapeutic activity.

Due to long time antibiotic treatment, acne patients often show gram-negative bacterial colonization on anterior nares. Usually the gram-negative bacterial population in normal subjects is below 1% of nasal bacterial population but, in the case of gram-negative folliculitis, it increases to more than 3-4%. While ampicillin is choice of antibiotic conventionally used against gram-negative bacteria, due to increasing antibiotic resistance in gram negative bacterial strains towards multiple drugs, cure of disease caused by such resistant organism is difficult. Accordingly, when tested, besifloxacin shows promising results.

Thus, further, and equally importantly, the MIC of besifloxacin is also considerably low against such gram-negative bacteria which are resistant or susceptible to other conventional antibiotic such as cefotaxime, ampicillin, amoxicillin, cephalosporin, clindamycin, tetracycline and erythromycin.

Therefore, all the bacterial species which are known to be involved in gram-negative folliculitis, are susceptible to besifloxacin, irrespective of their response against other conventional antibiotics or quinolones.

The MIC of besifloxacin against gram-negative bacteria selected from Escherichia coli, Klebsiella spp., Pseudomonas spp. Serratia spp., Acinetobacter spp., Enterobacter spp. and Proteus spp., is lower by at least 50% when compared with

MIC of at least one antibiotic selected from cefotaxime, ampicillin, clindamycin, tetracycline and erythromycin.

Further, none of the isolates of skin resident bacteria in acne patients or healthy individuals demonstrate resistance to besifloxacin in micro broth dilution assay. Moreover, as the MIC values are low and within a narrow range, the chance of future resistance generation due to sub optimal drug delivery in skin is also substantially lower.

Additional embodiments and features of the present disclosure will be apparent to one of ordinary skill in art based upon description provided herein. The embodiments herein and the various features and advantageous details thereof are explained with reference to the nonlimiting embodiments in the description. Descriptions of well-known/conventional methods and techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the following examples should not be construed as limiting. The examples are illustrative only, and are not intended to limit, in any manner, any of the aspects described herein. The following examples do not in any way limit the invention.

### EXAMPLES

### Example 1A. Minimum Inhibitory Concentration (MIC) of Besifloxacin

MIC of besifloxacin and other antibiotics molecules were determined by micro broth dilution method as per the Clinical and Laboratory Standards Institute (CLSI) guideline. MIC assay is the determinant of the inherent anti-microbial potency of a particular molecule. For preparing inoculum, bacterial strains were cultured in Brain Heart Infusion Agar (BHIA) at 37° C. for 24 hours. For MIC test, sterile BHI broth (100 μl) was added into all 96 wells and 100 μl of broth containing drug was added to first well (1A to 1H) and serial (double) dilution was carried out for up to 10 wells (column 1 to column 10 of 96 well plate). For bacterial inoculum, bacterial culture turbidity was adjusted to 0.1 OD at 600 nm in UV-Visual spectrophotometer (approximately 1.5×10⁸ cells/ml) and further diluted (100 times with sterile BHI broth). Diluted bacterial suspension (100 μl) was added to each well except sterility control wells (column 12 of 96 well plate). Column 11 of 96 well plate was used as growth control and vehicle control. The plates were incubated at 37° C. for 24 h. The MIC of the test compounds were determined by observing the lowest concentration of test compound that prevented the visual bacterial growth. Interestingly all the bacterial species which are known to be involved in gram negative folliculitis, are susceptible to besifloxacin irrespective of their response against other conventional antibiotics or Quinolones (Table 1).

TABLE 1A
MIC of besifloxacin tested against key
bacterial strains involved in GNF

| S. | | MIC values |
|---|---|---|
| No. | Organisms | (μg/ml) |
| 1 | E. coli MTCC 1687 (wild type) | 0.125 |
| 2 | E. coli ATCC BAA 196 (multi-drug resistant strain) | 1 |
| 3 | K. pneumoniae ATCC 13883 (wild type) | 0.25 |
| 4 | P. aeruginosa ATCC 27853 (wild type) | 0.5 |
| 5 | P. aeruginosa CCARM 2204 (multi-drug resistant, | 4.0 |
| | quinolone susceptible) | |
| 6 | A. haumannii ATCC 19606 (wild type) | 1.0 |
| 7 | A. baumannii CCARM 12020 (multi-drug resistant, | 4.0 |
| | quinolone susceptible) | |
| 8 | A. baumannii CCARM 12001 (Quinolone | 2.0 |
| | resistant strain) | |
| 9 | E. aerogenes NCIM 5139 (wild type) | 0.125 |
| 10 | E. aerogenes M2-1 (clinical isolate) | 0.125 |

### Example 1B. Minimum Inhibitory Concentration (MIC) of Besifloxacin

Besifloxacin HCL was tested against ampicillin and cefotaxime (3rd generation cephalosporin) susceptible and resistant gram-negative bacterial strains involved in GNF (gram negative folliculitis) by broth dilution method as per the Clinical & Laboratory Standards Institutes (CLSI) guidelines. MIC assay was performed in 96 well plates by micro broth dilution method, in 96 well plates, all the wells were added with 100 μl of sterilized Muller Hinton broth No: 2 cation broth (HiMedia, India). The initial stock of test compounds (1 mg/ml) were prepared in dimethyl sulfoxide (DMSO) and further dilutions were made in culture broth to get the desirable concentration. Then 100 μl of broth containing test compounds were initially added to the first column's wells and then serial dilution was performed up to well of 10th column. Wells of 11th and 12th column served as a positive and negative control respectively. Finally, 100 μl of inoculum (0.5 McFarland equal bacterial suspension was 100 times diluted in culture broth) was added to all the wells. Plates were incubated at 37° C. for 18-24 hrs. After incubation, MIC of each test compound was determined by observing the lowest concentration of test compound that has no visual growth compared to growth control. Minimum inhibitory concentration of besifloxacin, ampicillin and cefotaxime against gram negative bacterial strains were shown in Table 1. Besifloxacin was very effective against both susceptible (S) and resistant (R) (ampicillin and cefotaxime) gram negative bacterial strains (Table 1B).

TABLE 1B
Minimum inhibitory concentration of Besifloxacin
against susceptible and ampicillin- and cefotaxime-
resistant gram-negative bacterial strains.
Minimum Inhibitory Concentration (μg/ml)

| Bacterial strains | Cefotaxime | Ampicillin | Besifloxacin |
|---|---|---|---|
| Escherichia coli 591 | <0.25 | 4 | 0.125 |
| Escherichia coli 592 | 128 | >128 | 0.5 |
| Klebsiella sp. 512 | <0.25 | 32 | 0.25 |
| Klebsiella sp. 498 | 64-128 | >128 | 16 |
| Pseudomonas sp. 556 | 4 | >128 | 1 |
| Pseudomonas sp. 537 | 16 | >128 | 1 |
| Enterobacter sp. 523 | 128 | >128 | 0.5 |
| Enterobacter sp. 532 | 64 | >128 | 16 |
| Enterobacter sp. 1 | 128 | >128 | 16 |
| Enterobacter sp. 2 | 0.5 | 2 | 1 |
| Acinetobacter sp. 488 | >128 | >128 | 2 |

Break Points- EUCAST 2019 Guide lines

| | |
|---|---|
| Cefotaxime | Ampicillin |

| Bacterial Stains | S | R | S | R |
|---|---|---|---|---|
| Escherichia coli | ≤1 | ≥2 | ≤8 | ≥8 |
| Klebsiella sp. | ≤1 | ≥2 | ≤8 | ≥8 |

| | | |
|---|---|---|
| Pseudomonas sp. | Not applicable | Not applicable |

| | | | | |
|---|---|---|---|---|
| Enterobacter sp. | ≤1 | ≥2 | ≤8 | ≥8 |

| | | |
|---|---|---|
| Acinetobacter sp. | Not applicable | Not applicable |

Due to long time antibiotic treatment, acne patients often show gram negative bacterial colonization on anterior nares. Usually the gram-negative bacterial population in normal people is below 1% of nasal bacterial population but, in the case of gram-negative folliculitis, it increases to more than 3-4%. Gram-negative bacteria include Escherichia coli, Klebsiella, Enterobacter, Proteus and Pseudomonas, which were isolated from infective persons. Ampicillin is the antibiotic of choice used for GNF treatment. However, due to increasing antibiotic resistance in gram negative bacterial strains towards multiple drugs, GNF is difficult to cure. Hence besifloxacin has been tested against various gram-negative bacterial strains susceptible and resistant to ampicillin and cefotaxime. As per the break points provided by EUCAST, some of these isolates showed higher MICs against ampicillin and cefotaxime. Besifloxacin was found to be efficacious when tested against these isolates.

### Example 1C. Minimum Inhibitory Concentration (MIC) of Besifloxacin

Different skin resident bacteria were isolated from acne lesions of moderate to severe acne patients. As expected, P. acnes and S. epidermidis were two most prevalent species. Members of the skin microbiomes were of diverse nature. Most of the genus were gram positive, but few of them, like Klebsiella, were gram negative. Most of them were aerobic, but few, like P. acnes, were anaerobic in nature. Irrespective of these differences and their prevalence, it was important to evaluate the MIC of besifloxacin vis a vis other conventionally used antibiotics in acne (clindamycin, tetracycline, erythromycin) and fluoroquinolones against these species. Even if they are present in low numbers in microbiomes, they can act as reservoirs of resistance against antibiotic of choice which may in the log run affect the prognosis of GNF. Besifloxacin has shown potent antibacterial activity against all of them (Table 2).

TABLE 2
Besifloxacin activity against bacteria isolated from acne lesions.
Minimum Inhibitory Concentration (μg/ml)

| S. | | Clinda- | Tetra- | Erythro- | Besi- |
|---|---|---|---|---|---|
| No. | Strain Name | mycin | cycline | mycin | floxacin |
| 1 | P. acnes V3-1 | 0.5 | 0.3 | >150 | 0.5 |
| 2 | S. epidermidis S1-1 | >4 | >4 | >4 | 0.25 |
| 3 | P. avidum S3-1 | 0.06 | 1 | 0.06 | 0.5 |
| 4 | K. pneumoniae S6-1 | >4 | >4 | >4 | 1 |
| 5 | Dermatobacter hominis | >4 | >4 | >4 | 1 |
| | S11-2 | | | | |
| 6 | Kocuria sp. S18-1 | 1 | 1 | >4 | 0.5 |
| 7 | Microbacterium sp. S20-2 | >4 | 2 | 0.25 | 0.5 |
| 8 | Blastococcus sp. S21A-2 | 0.25 | 0.5 | <0.01 | 0.06 |
| 9 | Staphylococcus | 0.13 | 0.25 | 0.25 | 0.13 |
| | haemolyticus M1-1 | | | | |
| 10 | Bacillus megaterium M1-2. | >4 | 0.06 | 0.25 | 0.02 |
| 11 | Enterobacter aerogenes | >4 | 2 | >4 | 0.25 |
| | M2-1 | | | | |

### Example 2: Preparation of Besifloxacin HCl Loaded Gel Formulations with Different Concentrations of Carbomer and Hydroxyethyl Cellulose

Gel formulations containing suspended besifloxacin is prepared using different concentrations of thickening agents as per the compositions shown in Table 3. The formulations had pH of 5.0-6.5.

TABLE 3
Gel Formulations Loaded with Suspended Besifloxacin•HCl for Compositions GL1 to GL8
using varying concentrations of Carbomer and Hydroxyethyl Cellulose as Thickening Agents

| | Composition of Besifloxacin Topical Gel (% w/w) | | | | | | | |
|---|---|---|---|---|---|---|---|---|
| Ingredients | GL1 | GL2 | GL3 | GL4 | GL5 | GL6 | GL7 | GL8 |
| Glycerin | 5.0 | 5.0 | 5.0 | 5.0 | 5.0 | 5.0 | 5.0 | 5.0 |
| Besifloxacin HCl | 1.09 | 1.09 | 1.09 | 1.09 | 1.09 | 1.09 | 1.09 | 1.09 |
| Purified water | q.s | q.s | q.s | q.s | q.s | q.s | q.s | q.s |
| | to 100 | to 100 | to 100 | to 100 | to 100 | to 100 | to 100 | to 100 |
| Edetate disodium | 0.1 | 0.1 | 0.1 | 0.1 | 0.1 | 0.1 | 0.1 | 0.1 |
| Hydroxyethyl | 1.2 | 1.2 | 1.2 | 0 | 1 | 0.8 | 0.6 | 0.4 |
| cellulose | | | | | | | | |
| Sodium | 0.2 | 0.2 | 0.2 | 0.2 | 0.2 | 0.2 | 0.2 | 0.2 |
| hyaluronate | | | | | | | | |
| Carbomer 980 | 0.1 | 0.3 | 0.5 | 0.85 | 0.5 | 0.5 | 0.5 | 0.6 |
| Phenoxyethanol | 0.7 | 0.7 | 0.7 | 0.7 | 0.7 | 0.7 | 0.7 | 0.7 |
| Polyethylene | 5.0 | 5.0 | 5.0 | 5.0 | 5.0 | 5.0 | 5.0 | 5.0 |
| glycol 400 | | | | | | | | |
| Diethylene glycol | 5.0 | 5.0 | 5.0 | 5.0 | 5.0 | 5.0 | 5.0 | 5.0 |
| monoethyl ether | | | | | | | | |
| Sod. Hydroxide | q.s. | q.s. | q.s. | q.s. | q.s. | q.s. | q.s. | q.s. |
| (18% w/w) | to pH | to pH | to pH | to pH | to pH | to pH | to pH | to pH |
| | 5.5-6.5 | 5.5-6.5 | 5.5-6.5 | 5.5-6.5 | 5.5-6.0 | 5.5-6.0 | 5.5-6.0 | 5.5-6.0 |

Characterization of Gel Formulations:

| Batch Code | GL1 | GL2 | GL3 | GL4 | GL5 | GL6 | GL7 | GL8 |
|---|---|---|---|---|---|---|---|---|
| pH | 6.28 | 6.33 | 6.32 | 5.5-6.0 | 5.58 | 5.72 | 5.93 | 5.73 |
| Yield Stress (Pa) | 3.78E−8 | 5.46E−6 | 3.95E−3 | 0 | 2.25E−06 | 4.18E−08 | 1.01E−06 | 7.98E−07 |
| Viscosity mPa · s* | 6900 | 10400 | 14500 | 4500 | 10700 | 7480 | 7200 | 6370 |

*Viscosity was measured using Anton Paar viscometer

### Method of Preparation:

1. In a main mixing vessel, edetate disodium is dissolved in purified water.
2. Then vortex is created under overhead stirring at 300 rpm, and thickening agent hydroxyethyl cellulose is added slowly to avoid any lump formation. The contents of the beaker are stirred to obtain a lump free dispersion.
3. In a separate vessel, carbomer is allowed to swell in purified water. The contents of the beaker are stirred for 30 min at 350 rpm using overhead stirrer till clear transparent phase is obtained. And neutralized with sodium hydroxide solution.
4. In a separate vessel, purified water is loaded and sodium hyaluronate is added under stirring and allowed to swell for 1 hour.
5. In a vessel, glycerin is loaded. Besifloxacin hydrochloride is slowly added to it under stirring at 300 rpm. Then the dispersion is stirred, followed by addition of sodium hydroxide solution. The mixture is allowed to stir further for 15 minutes. Now, drug dispersion is ready to be transfer to main mixing vessel.
6. Processing in main mixing vessel
   6.1. The content of carbomer phase is transferred to main mixing vessel containing hydroxyethyl cellulose phase and mixed for 20 minutes using overhead stirrer at 250 rpm.
   6.2. This is followed by addition of swelled sodium hyaluronate phase. The contents in the main mixing vessel are stirred for 5 minutes at 250 rpm using overhead stirrer.
   6.3. Then phenoxyethanol is directly added to the main mixing vessel and mixed for 30 minutes at 250 rpm using overhead stirrer.
   6.4. The besifloxacin phase is then added to the main mixing vessel and mixed.
   6.5. Polyethylene glycol 400, diethylene glycol monoethyl ether is added to the main mixing vessel.
   6.6. pH of resulting gel is adjusted to 5.5-6.5 using sodium hydroxide solution.
   6.7. The contents in the main mixing vessel are stirred using overhead stirrer for additional 2 hr. A white homogenous gel is obtained.

### Example 3: Preparation of Besifloxacin HCl Loaded Gel Formulations

Gel formulations containing suspended besifloxacin using various thickening agents are prepared as per the compositions shown in Table 4. The formulations had pH of 5.0-6.5.

TABLE 4
Gels Loaded with Suspended Besifloxacin•HCl for Compositions
GL9, GL10, GL11, GL12, GL13, and GL14 using Carbomer,
Hydroxyethyl Cellulose, Sepineo as Thickening Agents

| | Composition (% w/w) | | | | | |
|---|---|---|---|---|---|---|
| Ingredients | GL9 | GL10 | GL11 | GL12 | GL13 | GL14 |
| Besifloxacin•HCl | 1 | 2 | 4 | 1 | 2 | 1 |
| equivalent to | | | | | | |
| besifloxacin | | | | | | |
| Edetate disodium | 0.1 | 0.1 | 0.1 | 0.1 | 0.1 | 0.1 |
| dihydrate | | | | | | |
| Carbomer | 0.65 | 0.65 | 0.65 | 0.65 | 0 | 0.3 |
| homopolymer type C | | | | | | |
| Diethylene glycol | 5 | 5 | 5 | 0 | 2 | 1 |
| monoethyl ether | | | | | | |
| Polyethylene glycol | 5 | 5 | 5 | 3 | 4 | 5 |
| 400 | | | | | | |
| Hydroxyethyl | 0.6 | 0.6 | 0.6 | 0.6 | 0 | 0 |
| cellulose | | | | | | |
| Sepineo P600 | 0 | 0 | 0 | 0 | 5 | 4 |
| Sodium hyaluronate | 0.2 | 0.2 | 0.2 | 0.2 | 0.2 | 0 |
| Phenoxyethanol | 0.7 | 0.7 | 0.7 | 0.7 | 0.7 | 0.7 |
| Glycerin | 5 | 5 | 5 | 5 | 5 | 5 |
| Sodium hydroxide | q.s. | q.s. | q.s. | q.s. | q.s. | q.s. |
| solution | to PH | to PH | to PH | to PH | to PH | to PH |
| Purified water | q.s. | q.s. | q.s. | q.s. | q.s. | q.s. |
| | to 100 | to 100 | to 100 | to 100 | to 100 | to 100 |

Characterization of Gels:

| | GL9 | GL10 | GL11 | GL12 | GL13 | GL14 |
|---|---|---|---|---|---|---|
| pH | 6.31 | 6.15 | 6.07 | 5.7 | 5.0 | 5.1 |
| Viscosity | 10300 | 8123 | 7169 | 8320 | 6500 | 9832 |

### Method of Preparation:

1. In a main mixing vessel, purified water is loaded and edetate disodium is added to it under overhead stirring at 250 rpm. The mixture is stirred till clear solution is obtained.
2. Then vortex is created under overhead stirring at 300 rpm, and thickening agent(s) carbomer homopolymer type C, and/or Sepineo is added slowly to avoid any lump formation. The contents of the beaker are stirred to obtain a lump free dispersion.
3. In a separate vessel, hydroxyethyl cellulose is allowed to swell in purified water. Followed by addition of solvents such as diethylene glycol monoethyl ether and/or polyethylene glycol 400. The contents of the beaker are stirred for 1 hour at 350 rpm using overhead stirrer till clear transparent phase is obtained.
4. In a separate vessel, purified water is loaded and sodium hyaluronate is added under stirring and allowed to swell for 1 hour.
5. In a vessel, glycerin is loaded. Besifloxacin hydrochloride is slowly added to it under stirring at 300 rpm. Then the dispersion is stirred for 15 minutes. Sodium hydroxide solution is added in three consecutive portions with 5 minutes stirring at 300 rpm after each addition. The mixture is allowed to stir further for 15 minutes. Now, drug dispersion is ready to be transfer to main mixing vessel.
6. Processing in main mixing vessel
   6.1. The content of hydroxyethyl cellulose phase is transferred to main mixing vessel containing Carbomer phase and/or Sepineo and mixed for 20 minutes using overhead stirrer at 250 rpm.
   6.2. This is followed by addition of swelled sodium hyaluronate phase. The contents in the main mixing vessel are stirred for 5 minutes at 250 rpm using overhead stirrer. Then phenoxyethanol is directly added to the main mixing vessel and mixed for 30 minutes at 250 rpm using overhead stirrer. pH of the resulting mixture is adjusted to 6.0 to 6.5 using sodium hydroxide solution. The mixture is stirred till it becomes thick and viscous.
   6.3. The besifloxacin phase is then added to the main mixing vessel and stirred for 45 min at 350 rpm.
   6.4. The contents in the main mixing vessel are stirred using overhead stirrer for additional 1 hour at 350 rpm. The quantity of water (q.s. to 100%) is made up and the mixture is stirred for 1 h at 350 rpm under overhead stirrer. A white homogenous gel is obtained.

### Example 4: Preparation of Besifloxacin HCl Loaded Gel Formulations with Different Active Strengths

Gel formulations containing suspended besifloxacin are prepared as per the compositions shown in Table 5. The formulations prepared is off-white to pale yellow in appearance with a pH of 5.0-6.5.

TABLE 5
Gel Formulations Loaded with Suspended Besifloxacin•HCl for
Compositions GL15, GL16, GL17, GL18, GL19, GL20, GL21, GL22 and GL23

| | | Composition of Besifloxacin Gels (% w/w) | | | | | | | | |
|---|---|---|---|---|---|---|---|---|---|---|
| Phase | Ingredients | G15 | G16 | G17 | G18 | G19 | G20 | G21 | G22 | G23 |
| A | Purified | q.s. | q.s. | q.s. | q.s. | q.s. | q.s. | q.s. | q.s. | q.s. |
| | water | | | | | | | | | |
| | Edetate | 0.10 | 0.10 | 0.1 | 0.1 | 0.1 | 0.1 | 0.1 | 0.1 | 0.1 |
| | disodium | | | | | | | | | |
| | Allantoin | 0.20 | 0.20 | 0.2 | 0.2 | 0.2 | 0 | 0 | 0 | 0 |
| | Hydroxyethyl | 0 | 0 | 0.9 | 0.9 | 0.9 | 0.6 | 0.6 | 0.6 | 0.6 |
| | cellulose | | | | | | | | | |
| | Carbomer | 0.85 | 0.85 | 0 | 0 | 0 | 0.65 | 0.65 | 0.65 | 0.65 |
| | 980 | | | | | | | | | |
| B | Purified | 10 | 10 | 42.25 | 42.25 | 42.25 | 10 | 10 | 10 | 10 |
| | water | | | | | | | | | |
| | Sodium | 0.40 | 0.40 | 0.4 | 0.4 | 0.4 | 0.2 | 0.2 | 0.1 | 0.1 |
| | hyaluronate | | | | | | | | | |
| C | Poloxamer | 0 | 0 | 0 | 0 | 0 | 0 | 0 | 0 | 0.2 |
| | 407 | | | | | | | | | |
| D | Phenoxyethanol | 0.70 | 0.70 | 0.7 | 0.7 | 0.7 | 0.7 | 0.7 | 0.7 | 0.7 |
| | Sodium | 1.20 | 1.20 | 0.04 | 0.04 | 0.04 | 0.5 | 0.5 | 0.5 | 0.5 |
| | hydroxide | | | | | | | | | |
| | (18% w/v) | | | | | | | | | |
| E | Glycerin | 5.00 | 5.00 | 5 | 5 | 5 | 3 | 3 | 2 | 1 |
| | Besifloxacin | 1.0 | 1.5 | 1.0 | 2.0 | 4.0 | 2.5 | 3.0 | 3.5 | 0.5 |
| | HCl equivalent to | | | | | | | | | |
| | Besifloxacin | | | | | | | | | |
| | Purified | 38.44 | 38.44 | 38.89 | 38.89 | 38.89 | 20 | 20 | 20 | 20 |
| | water | | | | | | | | | |
| | Sodium | q.s | q.s | q.s | q.s | q.s | q.s | q.s | q.s | q.s |
| | hydroxide | to pH | to pH | to pH | to pH | to pH | to pH | to pH | to pH | to pH |
| | solution | | | | | | | | | |
| F | Polyethylene | 5.00 | 5.00 | 5 | 5 | 5 | 5 | 5 | 5 | 5 |
| | glycol 400 | | | | | | | | | |
| | Diethylene | 5.00 | 5.00 | 5 | 5 | 5 | 5 | 5 | 5 | 5 |
| | glycol | | | | | | | | | |
| | monoethyl | | | | | | | | | |
| | ether | | | | | | | | | |
| G | Sodium | q.s. | q.s. | q.s. | q.s. | q.s. | q.s. | q.s. | q.s. | q.s. |
| | hydroxide | to pH | to pH | to pH | to pH | to pH | to pH | to pH | to pH | to pH |
| | (18% w/v) | 5.5-6.0 | 5.5-6.0 | 5.5-6.5 | 5.5-6.5 | 5.5-6.5 | 5.5-6.5 | 5.5-6.5 | 5.5-6.5 | 5.5-6.5 |

| | Characterization of Gel Formulations | | | | | | | | |
|---|---|---|---|---|---|---|---|---|---|
| Batch Code | G15 | G16 | G17 | G18 | G19 | G20 | G21 | G22 | G23 |
| pH | 5.5 | 5-5.5 | 5.5 | 5.5 | 5.5 | 5.8 | 5.6 | 5.8 | 5.5 |
| Viscosity mPa · s* | 4322 | 3582 | 4028 | 4198 | 3842 | 5300 | 4800 | 4534 | 4019 |

| | |
|---|---|
| Appearance | White to pale yellow gel |

*Viscosity was measured using Anton Paar viscometer

### Method of Preparation:

1) In a main mixing vessel, allantoin is dissolved in hot water (heated up to 50° C.).
2) Then, allantoin solution is allowed to cool to room temperature.
3) Carbomer and/or hydroxyethyl cellulose is added to above water and allowed to swell for 1.0 hr.
4) In a separate vessel, sodium hyaluronate is allowed to swell in water for 1.0 hr.
5) Sodium hyaluronate phase is added to carbomer/hydroxyethyl cellulose phase and mixed.
6) In a separate vessel, Polaxomer 407 is dissolved in water, and added to swelled carbomer phase in step 5 and mixed.
7) Besifloxacin is dispersed in water and transferred to carbomer phase of step 4.
8) Remaining raw materials, glycerin, polyethylene glycol 400, disodium edetate, phenoxyethanol, and diethylene glycol monoethyl ether are added to the carbomer phase in sequence with 5-minute intermittent gap with continuous mixing. The bulk is finally mixed for 15 min.
9) pH of resulting formulation is adjusted to 5.0-5.5 with triethanolamine.
10) The resulting formulation is mixed for 1.0 hr.

### Example 5: Preparation of Besifloxacin HCl Loaded Gel Formulations with Carbomer 980

Gel formulations containing suspended besifloxacin are prepared using carbomer 980 as per the compositions shown in Table 6. The formulations prepared is off-white to pale yellow in appearance with a pH of 5.0-6.5. Method of preparation is same as given in example 3.

TABLE 6
Gel Formulations Loaded with Suspended Besifloxacin•HCl
for Compositions GL24, GL25, and GL26

| | Composition (% w/w) | | |
|---|---|---|---|
| Chemical Name | GL24 | GL25 | GL26 |
| Purified water | q.s. to 100 | q.s. to 100 | q.s. to 100 |
| Allantoin | 0.2 | 0.2 | 0.2 |
| Carbomer 980 | 0.85 | 0.85 | 0.85 |
| Sodium hyaluronate | 0.4 | 0.4 | 0.4 |
| Poloxamer 407 | 0.2 | 0.2 | 0.2 |
| Besifloxacin HCl | 1.09 | 1.09 | 1.09 |
| Glycerin | 5.00 | 5.00 | 5.00 |
| Polyethylene glycol 400 | 7.00 | 5.00 | 5.00 |
| Edetate Disodium | 0.10 | 0.10 | 0.10 |
| Phenoxyethanol | 0.50 | 0.50 | 0.50 |
| Triethanolamine | 1.00 | 1.00 | 1.00 |
| Diethylene glycol monoethyl | 0 | 0 | 5 |
| ether | | | |

### Example 6: Preparation of Besifloxacin HCl Loaded Cream Formulations

Cream formulations containing suspended besifloxacin are prepared as per the compositions shown in Table 7. The formulations had pH of 5.0-6.5.

TABLE 7
Cream Formulations Loaded with Suspended Besifloxacin•HCl
for Compositions CR1 and CR2.

| | Composition (% w/w) | |
|---|---|---|
| Ingredients | CR1 | CR2 |
| Phase A | | |
| Pemulen TR1 | 0.4 | 0.3 |
| Cyclopentasiloxane | 5.0 | 5.0 |
| Cetyl Alcohol | 2.0 | 1.0 |
| Stearyl Alcohol | 2.0 | 1.0 |
| Steareth 2 | 2.0 | 2.0 |
| Phase B | | |
| Besifloxacin•HCl equivalent to | 1.0 | 1.0 |
| Besifloxacin | | |
| Propylene Glycol | 5.0 | 5.0 |
| Steareth 21 | 2.0 | 2.0 |
| Water | q.s. to 100 | q.s. to 100 |
| Sodium hydroxide (10%) | q.s. to Ph 5.5-6.0 | q.s. to Ph 5.5-6.0 |
| Phase C | | |
| BHT | 0 | 0.1 |
| Phenonip | 0 | 0.5 |

### Method of Preparation:

1) Cyclopentasiloxane, cetyl alcohol, stearyl alcohol and steareth 2 are heated at 50° C. (Phase A).
2) In a separate vessel (Phase B), Steareth 21 is solubilized in water with stirring at 500 rpm and heating at 50° C.
3) After complete solubilization of steareth 21, propylene glycol and besifloxacin are added.
4) Then, sodium hydroxide solution is added to get transparent solution (Phase B).
5) Phase A is heated to 50° C. and then pemulen is dispersed.
6) Phase B is heated separately to 50° C.
7) Then, Phase A is added to Phase B with stirring at 500 rpm.
8) Resulting formulation is cooled to room temperature with stirring.
9) Phase C components are added to step 8, if applicable. Mixed.
10) The pH of resulting cream is recorded.

### Example 7: Preparation of Cream Formulations Containing Besifloxacin and its Combination with Isotretinoin or Tretinoin

Cream formulations containing suspended actives such as besifloxacin alone and/or combination of besifloxacin and isotretinoin and/or besifloxacin and tretinoin are prepared as per the compositions shown in Table 8. The formulations had pH of 5.0-6.5.

TABLE 8
Cream Formulations Loaded with Besifloxacin alone, and Besifloxacin and Retinoids
such as Isotretinoin and Tretinoin for Compositions CR3, CR4, CR5, CR6, CR7,
CR8, CR9, CR10 and CR11 Prepared using Carbomer as Thickening Agents

| | Composition (% w/w) | | | | | | | | |
|---|---|---|---|---|---|---|---|---|---|
| Ingredients | CR3 | CR4 | CR5 | CR6 | CR7 | CR8 | CR9 | CR10 | CR11 |
| Phase A | | | | | | | | | |
| Cyclomethicone | 5 | 6 | 5 | 5 | 5 | 5 | 5 | 5 | 5 |
| Cetyl alcohol | 1 | 1 | 1 | 1 | 1 | 1 | 2 | 2 | 2 |
| Stearyl alcohol | 1 | 1 | 1 | 1 | 1 | 2 | 1 | 1 | 1 |
| Steareth 2 | 2 | 2 | 2 | 2 | 2 | 2 | 2 | 2 | 2 |
| Steareth 21 | 2 | 2 | 2 | 2 | 2 | 2 | 2 | 2 | 2 |
| Light liquid | 3 | 3 | 3 | 3 | 2.5 | 3.5 | 3 | 3 | 3 |
| paraffin | | | | | | | | | |
| Phase B | | | | | | | | | |
| Besifloxacin•HCl | 1 | 2 | 1.5 | 4 | 1 | 0.5 | 2 | 0.5 | 2 |
| equivalent to | | | | | | | | | |
| Besifloxacin | | | | | | | | | |
| Sodium | 1.0 | 2.0 | 1.0 | 1.0 | 1.0 | 1.0 | 2.0 | 2.0 | 2.0 |
| hydroxide | | | | | | | | | |
| solution (10%) | | | | | | | | | |
| Water | 40.41 | 50 | 50 | 50 | 50 | 50 | 50 | 50 | 50 |
| Glycerol | 0 | 0 | 0 | 5.0 | 2 | 3 | 5 | 5 | 5 |
| Phase C | | | | | | | | | |
| Glycerol | 5.0 | 5.0 | 5.0 | 0 | 0 | 0 | 0 | 0 | 0 |
| Carbopol 980 | 15 | 20 | 20 | 25 | 25 | 25 | 25 | 25 | 25 |
| (2%) | | | | | | | | | |
| Sodium | 1.0 | q.s. | 1.0 | 1.0 | 1.0 | 1.0 | 1.0 | 1.0 | 1.0 |
| hydroxide | | to pH | | | | | | | |
| solution (10%) | | | | | | | | | |
| Water | q.s. | q.s. | q.s. | q.s. | q.s. | q.s. | q.s. | q.s. | q.s. |
| | to 100 | to 100 | to 100 | to 100 | to 100 | to 100 | to 100 | to 100 | to 100 |
| Phase D | | | | | | | | | |
| Isotretinoin | 0 | 0 | 0 | 0 | 0.05 | 0.05 | 0.1 | 0 | 0 |
| Tretinoin | 0 | 0 | 0 | 0 | 0 | 0 | 0 | 0.1 | 0.025 |
| Poloxamer 407 | 0 | 0 | 0 | 0 | 0.1 | 0.1 | 0.1 | 0.1 | 0.1 |
| Water | 0 | 0 | 0 | 0 | 10 | 10 | 10 | 10 | 10 |
| PEG 400 | 0 | 0 | 0 | 0 | 0 | 10 | 0 | 10 | 5 |
| Phase E | | | | | | | | | |
| Butylated | 0.1 | 0 | 0.1 | 0.1 | 0 | 0 | 0 | 0 | 0 |
| Hydroxy Toluene | | | | | | | | | |
| Phenoxyethanol | 0.7 | 0.7 | 0.7 | 0.7 | 0.7 | 0.7 | 0.7 | 0.7 | 0.7 |

Observation:
Off-white to pale yellow, homogeneous cream

### Method of Preparation:

1) Cyclopentasiloxane, cetyl alcohol, stearyl alcohol, steareth 2, steareth 21 and light liquid paraffin are heated at 65° C. (Phase A).
2) In another vessel, besifloxacin is dispersed in water containing sodium hydroxide and heated to 65° C. (Phase B) in a main mixing vessel.
3) Then, Phase A is added to Phase B with continuous stirring at 600 rpm.
4) Step 3 is slowly cooled to 40° C.
5) In a vessel, glycerol, Carbopol and water are mixed together. Then neutralized with sodium hydroxide solution (Phase C).
6) Phase C is added to step 4 and mixed.
7) In a separate vessel (Phase D), suspension of isotretinoin or tretinoin is prepared. Briefly, poloxamer 407 is solubilized in purified water. Then PEG 400 and isotretinoin or tretinoin is added one by one and mixed. This dispersion is ready to be add to the main mixing vessel. Special precaution needs to be taken to protect the product from light exposure.
8) Dispersion of phase D is added to the main mixing vessel of step 6 and mixed. Special precaution needs to be taken to protect the product from light exposure.
9) Component of Phase E are added one by one to step 8. Mixed.
10) Resulting formulation is cooled to room temperature.

### Example 8: Preparation of Besifloxacin HCl Soluble Gel Formulations

Gel formulations containing soluble besifloxacin is prepared as per compositions shown in Table 9.

TABLE 9
Gel Formulations Loaded with Soluble Besifloxacin•HCl
for Compositions GL18, GL19 and GL20

| | Composition (% w/w) | | |
|---|---|---|---|
| Ingredients | GL27 | GL28 | GL29 |
| Phase A | | | |
| Besifloxacin•HCl equivalent to | 1 | 1 | 1 |
| Besifloxacin | | | |
| Sodium lauryl sulphate (30%) | 5 | 5 | 5 |
| Tween 80 | 8 | 8 | 8 |
| Diethylene glycol monoethyl ether | 16 | 10 | 16 |
| Propylene Glycol | 4 | 0 | 4 |
| Phase B | | | |
| Disodium edetate | 0.1 | 0.1 | 0.1 |
| Sodium hydroxide (10%) | 2 | 2 | 2 |
| Carbomer 980 (3%) | 25 | 25 | 25 |
| Water | q.s to 100 | q.s to 100 | q.s to 100 |
| Phase C | | | |
| Phenoxyethanol | 1 | 1 | 1 |
| Triethanolamine | 1 | 1 | 1 |

Observation:
Transparent, pale yellow gel

### Method of Preparation:

1) In main mixing vessel, disodium edetate is dissolved in purified water.
2) Carbomer is added in the main mixing vessel and mixed.
3) Component of step 2 is neutralized with sodium hydroxide solution.
4) In a side vessel, besifloxacin is dispersed in sodium lauryl sulphate, tween 80, diethylene glycol monoethyl ether and propylene glycol.
5) Component of step 4 are transferred to the main mixing vessel and mixed.
6) Phenoxyethanol is added to above vessel.
7) If necessary, adjust the pH of obtained gel to 5.5-6.5.

### Example 9: Preparation of Lotion Formulations Containing Besifloxacin

Lotion formulations of besifloxacin HCl are prepared as per compositions shown in Table 10.

TABLE 10
Lotion Formulations Containing Besifloxacin•HCl
for Compositions LO1, LO2, LO3, LO4, LO5 and LO6

| | Composition (% w/w) | | | | | |
|---|---|---|---|---|---|---|
| Ingredients | LO1 | LO2 | LO3 | LO4 | LO5 | LO6 |
| Phase A | | | | | | |
| PEG-400 stearate | 1 | 1 | 1 | 1 | 1 | 1 |
| Glyceryl stearate | 2 | 3 | 2 | 2.5 | 1.5 | 2 |
| Light liquid | 3 | 2 | 5 | 0 | 3 | 7 |
| paraffin | | | | | | |
| Behenyl Alcohol | 1.5 | 1.0 | 1.5 | 1.0 | 1.5 | 1 |
| Phase B | | | | | | |
| Glycerol | 4.0 | 5.0 | 3.0 | 2.0 | 2.0 | 5.0 |
| Propylene Glycol | 0 | 0 | 1 | 5 | 4 | 2.0 |
| Besifloxacin•HCl | 1 | 0.5 | 2 | 1.5 | 3 | 4 |
| equivalent to | | | | | | |
| Besifloxacin | | | | | | |
| Water | 20 | 20 | 20 | 20 | 20 | 20 |
| Sodium | q.s | q.s | q.s | q.s | q.s | q.s |
| hydroxide | | | | | | |
| Phase C | | | | | | |
| Carbopol 980 | 0.55 | 0.2 | 0 | 0.5 | 0 | 0 |
| Xanthan Gum | 0 | 0 | 0.5 | 0 | 0 | 0.6 |
| Sepineo P600 | 0 | 4 | 0 | 0 | 5 | 0 |
| Water | q.s. | q.s. | q.s. | q.s. | q.s. | q.s. |
| | to | to | to | to | to | to |
| | 100 | 100 | 100 | 100 | 100 | 100 |
| Phase D | | | | | | |
| BHT | 0.1 | 0 | 0 | 0 | 0.1 | 0 |
| Phenoxyethanol | 0.5 | 0.7 | 0.5 | 0.7 | 0.5 | 0.7 |

### Method of Preparation:

1) Ina main mixing vessel, component of phase A are heated together to 80° C.
2) In a side vessel, besifloxacin is dispersed in glycerol and/or propylene glycol and water with continuous mixing at 300 rpm for 10 minutes. Then dilute solution of sodium hydroxide is added drop-wise to adjust pH to about 5.5. Now, Phase B is ready to transfer in the main mixing vessel.
3) Components of Phase B is heated to 80° C.
4) Heated mixture of phase B is added to oil phase with continuous mixing in main mixing vessel at 80° C., 200 rpm and allowed to mix for 15 minutes.
5) The content of the main mixing vessel is allowed to air-cool with mixing to 45° C.
6) In another vessel, Carbopol and/or xanthan gum and/or Sepineo are allowed to swell in water for 2 h and its pH is adjusted to about 5.5 to 6 with sodium hydroxide solution, which is then added to the main mixing vessel and mixed and mix for 30 minutes.
7) Then transfer the remaining components BHT and phenoxyethanol are added to the main mixing vessel and mixed for 20 minutes.
8) The lotion's pH is adjusted to about 5.5 to 6.0, if required.

### Example 10: Preparation of Cream Base Foam Formulations Containing Besifloxacin and its Combination with Retinoids

Foam formulations of besifloxacin HCl are prepared as per compositions shown in Table 11.

TABLE 11
Foam Formulations Containing Besifloxacin•HCl and Combination with
Retinoids for Compositions FO1, FO2, FO3, FO4, FO5, FO6, FO7, FO8 and FO9.

| | Composition (% w/w) | | | | | | | | |
|---|---|---|---|---|---|---|---|---|---|
| Ingredients | FO1 | FO2 | FO3 | FO4 | FO5 | FO6 | FO7 | FO8 | FO9 |
| Phase A | | | | | | | | | |
| Cetyl alcohol | 1 | 1 | 1 | 1 | 1 | 1 | 1 | 1 | 1 |
| Glyceryl | 1.5 | 1.5 | 1.5 | 1.5 | 1.5 | 1.5 | 1.5 | 1.5 | 1.5 |
| monostearete | | | | | | | | | |
| Cocamidopropyl | 0.5 | 0.5 | 0.5 | 0.5 | 0.5 | 0.5 | 0.5 | 0.5 | 0.5 |
| betaine | | | | | | | | | |
| Steareth 2 | 2 | 2 | 2 | 2 | 2 | 2 | 2 | 2 | 2 |
| Steareth 21 | 3 | 2.5 | 2.5 | 3 | 3 | 2.5 | 2.5 | 3 | 3 |
| Polysorbate 60 | 2 | 3 | 4 | 2 | 2 | 2 | 2 | 2 | 2 |
| Phase B | | | | | | | | | |
| Glycerol | 4.0 | 5.0 | 3.0 | 2.0 | 2.0 | 5.0 | 5.0 | 2.0 | 2.0 |
| Propylene | 0 | 0 | 1 | 5 | 4 | 2.0 | 2.0 | 5 | 5 |
| Glycol | | | | | | | | | |
| Besifloxacin•HCl | 1 | 0.5 | 2 | 1.5 | 3 | 4 | 4 | 1.0 | 2.0 |
| equivalent to | | | | | | | | | |
| Besifloxacin | | | | | | | | | |
| Water | 20 | 20 | 20 | 20 | 20 | 20 | 20 | 20 | 20 |
| Sodium | q.s | q.s | q.s | q.s | q.s | q.s | q.s | q.s | q.s |
| hydroxide | to pH | to pH | to pH | to pH | to pH | to pH | to pH | to pH | to pH |
| | 5.5 | 5.5 | 5.5 | 5.5 | 5.5 | 5.5 | 5.5 | 5.5 | 5.5 |
| Phase C | | | | | | | | | |
| Carbomer 980 | 0.4 | 0.4 | 0.5 | 0.5 | 0.5 | 0.5 | 0.5 | 0.5 | 0.5 |
| Sodium | q.s | q.s | q.s | q.s | q.s | q.s | q.s | q.s | q.s |
| hydroxide | | | | | | | | | |
| Water | q.s. | q.s. | q.s. | q.s. | q.s. | q.s. | q.s. | q.s. | q.s. |
| | to 100 | to 100 | to 100 | to 100 | to 100 | to 100 | to 100 | to 100 | to 100 |
| Phase D | | | | | | | | | |
| Isotretinoin | 0 | 0 | 0 | 0 | 0 | 0 | 0.1 | 0 | 0 |
| Tretinoin | 0 | 0 | 0 | 0 | 0 | 0 | 0 | 0.025 | 0.1 |
| BHT | 0.1 | 0 | 0 | 0 | 0.1 | 0 | 0 | 0.1 | 0.1 |
| Phenoxyethanol | 0.5 | 0.7 | 0.5 | 0.7 | 0.5 | 0.7 | 0.7 | 0.5 | 0.7 |
| Total | 100 | 100 | 100 | 100 | 100 | 100 | 100 | 100 | 100 |
| Propellant | 15 | 20 | 20 | 15 | 15 | 15 | 15 | 15 | 25 |

### Method of Preparation:

1) Phase A is heated to 80° C.
2) Component of Phase B are mixed together and heated up to 80° C.
3) Phase B is transferred to Phase A with continuous stirring at 500 rpm and mixed for 30 minutes.
4) Then, allow the mixture to cool to room temperature.
5) Meanwhile in a suitable vessel, carbomer 980 is swelled with water and pH of the phase is neutralized to 5.5-6.5 using sodium hydroxide solution (Phase C).
6) Phase C is added to step 4 and mix for 30 minutes.
7) Phase D are added one by one and mixed for additional one hour.
8) Resulting formulation is packed with propellant in a suitable container.

### Example 11: Preparation of Besifloxacin Emulgel Formulations

Emulgel formulations of besifloxacin are prepared as per compositions shown in Table 12.

TABLE 12
Emulgel Formulations Containing Besifloxacin•HCl
for Compositions EG1 and EG2

| | Composition (% w/w) | |
|---|---|---|
| Ingredients | EG1 | EG2 |
| Phase A | | |
| Caproyl 90 | 5.0 | 5.0 |
| Cetyl Alcohol | 1.0 | 0 |
| Steareth 2 | 2.0 | 0 |
| Laureth 4 | 0 | 4.9 |
| Brij 35 | 0 | 5.1 |
| Phase B | | |
| Besifloxacin•HCl equivalent to | 1.0 | 3.5 |
| Besifloxacin | | |
| Steareth 21 | 2.0 | 0 |
| D-α-Tocopherol polyethylene | 5.0 | 4.0 |
| glycol 1000 succinate (TPGS) | | |
| Poloxamer 407 | 7.0 | 2.0 |
| Poloxamer 124 | 5.0 | 0 |
| Propylene Glycol | 0 | 5.0 |
| Purified water | 0 | q.s. to 100 |
| Phase C | | |
| Propylene Glycol | 3.0 | 0 |
| Purified water | 49.0 | 0 |
| Phase D | | |
| Ultrez 21 (0.5% w/w) | 20.0 | 20.0 |
| Phase E | | |
| Triethanolamine | q.s. | q.s. |
| Product Observation: | | |

| | |
|---|---|
| Description | Pale yellow, soft, homogenous gel with |
| | pH 5.0-5.5 |

### Method of Preparation:

1) Phase B and Phase C are heated separately at 70° C. to 75° C.
2) Phase C is added into phase B with continuous stirring at 500 rpm
3) Resulting dispersion is stirred for 1 hr to get uniform solution.
4) Phase A is heated separately at 70° C. to 75° C. and added slowly to step 3, with continuous stirring at 500 rpm
5) Resulting formulation is allowed to cool to 40° C.
6) Phase D is added and finally pH of the formulation is adjusted to 5.0-5.3 using phase E.

### Example 12: Preparation of Emulgel Formulation Loaded with Besifloxacin, and Combination of Besifloxacin with Retinoids (Isotretinoin and Tretinoin)

Emulgel formulations are prepared by addition of besifloxacin in oil phase as per compositions shown in Table 13.

TABLE 13
Emulgel Formulations Containing Besifloxacin•HCl and
Its Combination with Isotretinoin or Tretinoin for Compositions
EG3, EG4, EG5, EGT01, EGT02, EGT03, EGT04 and EGT05.

| | Composition (% w/w) | | | | | | | |
|---|---|---|---|---|---|---|---|---|
| Ingredients | EG3 | EG4 | EG5 | EGT01 | EGT02 | EGT03 | EGT04 | EGT05 |
| Phase A | | | | | | | | |
| Besifloxacin•HCl | 1.0 | 2.0 | 4.0 | 1 | 2.0 | 4.0 | 1 | 1 |
| equivalent to | | | | | | | | |
| Besifloxacin | | | | | | | | |
| Caproyl 90 | 5.0 | 4.0 | 4.0 | 5.0 | 4.0 | 4.0 | 5.0 | 5.0 |
| Cetyl Alcohol | 1.0 | 1.0 | 1.0 | 1.0 | 1.0 | 1.0 | 1.0 | 1.0 |
| Steareth 2 | 2.0 | 3.0 | 2.0 | 2.0 | 3.0 | 2.0 | 2.0 | 2.0 |
| Steareth 21 | 2.0 | 2.0 | 2.5 | 2.0 | 2.0 | 2.5 | 2.0 | 2.0 |
| D-α-Tocopherol | 5.0 | 5.0 | 5.0 | 5.0 | 5.0 | 5.0 | 5.0 | 5.0 |
| polyethylene glycol | | | | | | | | |
| 1000 succinate (TPGS) | | | | | | | | |
| Poloxamer 407 | 7.0 | 6.5 | 8.0 | 7.0 | 6.5 | 8.0 | 7.0 | 7.0 |
| Poloxamer 124 | 5.0 | 5.0 | 5.0 | 5.0 | 5.0 | 5.0 | 5.0 | 5.0 |
| Phase B | | | | | | | | |
| Propylene Glycol | 3.0 | 5.0 | 7.0 | 3.0 | 5.0 | 7.0 | 3.0 | 3.0 |
| Purified water | 63.91 | 63.91 | 63.91 | q.s | q.s | q.s | q.s | q.s |
| | | | | to 100 | to 100 | to 100 | to 100 | to 100 |
| Phase C | | | | | | | | |
| Ultrez 21 (2% w/w) | 5.0 | 5.0 | 5.0 | 5.0 | 5.0 | 5.0 | 5.0 | 5.0 |
| Phase D | | | | | | | | |
| Isotretinoin | 0 | 0 | 0 | 0.1 | 0.05 | 0.025 | 0 | 0 |
| Tretinoin | 0 | 0 | 0 | 0 | 0 | 0 | 0.1 | 0.05 |
| PEG 500 | 0 | 0 | 0 | 10 | 7 | 5 | 10 | 11 |
| Water | 0 | 0 | 0 | 10 | 10 | 10 | 10 | 10 |
| Phase E | | | | | | | | |
| Triethanolamine | q.s. | q.s. | q.s. | q.s. | q.s. | q.s. | q.s. | q.s. |
| Phenoxyethanol | 0 | 0 | 0 | 0.7 | 0.7 | 0.7 | 0.7 | 0.7 |
| Product Observation: | | | | | | | | |

| | |
|---|---|
| Description | Slight yellow, soft, homogenous gel with pH 5.0-5.5 |

### Method of Preparation:

1) Phase A and Phase B are heated separately at 70° C. to 75° C.
2) Phase A is added into phase B with continuous stirring at 500 rpm
3) Resulting formulation is allowed to cool to 40° C.
4) Phase C (pre-neutralized with triethanolamine) is added to step 3, slowly, with continuous stirring at 500 rpm
5) Stirring is continued for 30 minutes.
6) Components of Phase D are mixed and added to step 5. The resulting gel is mixed for 30 minutes. Precaution to be taken to avoid direct exposure of phase D to direct light.
7) Then phenoxyethanol is added and mixed. Finally, pH of the formulation is adjusted to 5.0-5.5 using triethanolamine.

### Example 13: Preparation of Besifloxacin Emulgel Formulations

Emulgel formulations of besifloxacin HCl are prepared by addition of besifloxacin in water phase as per compositions shown in Table 14.

TABLE 14
Emulgel Formulations Containing Besifloxacin•HCl
for Compositions EG6, EG7, EG8

| | Composition (% w/w) | | |
|---|---|---|---|
| Ingredients | EG6 | EG7 | EG8 |
| Phase A | | | |
| Besifloxacin•HCl equivalent to | 1.0 | 1.5 | 2.5 |
| Besifloxacin | | | |
| Steareth 21 | 2.0 | 2.0 | 2.0 |
| D-α-Tocopherol polyethylene | 5.0 | 4.0 | 3.0 |
| glycol 1000 succinate (TPGS) | | | |
| Poloxamer 407 | 7.0 | 5.0 | 7.0 |
| Poloxamer 124 | 5.0 | 7.0 | 5.0 |
| Propylene Glycol | 3.0 | 5.0 | 3.0 |
| Purified water | q.s. to 100 | q.s. to 100 | q.s. to 100 |
| Phase B | | | |
| Caproyl 90 | 5.0 | 5.0 | 4.0 |
| Cetyl Alcohol | 1.0 | 2.0 | 2.0 |
| Steareth 2 | 2.0 | 2.0 | 2.0 |
| Cyclomethicon | 1.0 | 1.0 | 1.0 |
| Phase C | | | |
| Ultrez 21 (1% w/w) | 10.0 | 12.0 | 15.0 |
| Phase D | | | |
| Triethanolamine | q.s. | q.s. | q.s. |
| Product Observation: | | | |

| | |
|---|---|
| Description | Slight yellow, soft, homogenous |
| | gel with pH 5.0-5.5 |

### Method of Preparation:

1) Phase A and Phase B are heated separately at 70° C. to 75° C.
2) Phase B is added to phase A with continuous stirring at 500 rpm
3) Resulting formulation is allowed to cool to 40° C.
4) Phase C is pre-neutralized with triethanolamine to pH 7.0 and added to step 3, slowly, with continuous stirring at 500 rpm
5) Stirring is continued for 30 minutes and finally pH of the formulation is adjusted to 5.0-5.5 using phase D.

### Example 14: Preparation of Besifloxacin and Retinoid (Isotretinoin and Tretinoin) Loaded Gel Formulations

Gel formulations containing besifloxacin and isotretinoin or tretinoin are prepared using different concentrations of thickening agents as per the compositions shown in Table 15. The formulations had pH of 5.0-6.5.

TABLE 15
Gel Formulations Compositions (TSG1 to TSG8) Loaded with Besifloxacin
and Isotretinoin or Tretinoin, prepared by varying concentrations
of Carbomer and Hydroxyethyl Cellulose as Thickening Agents

| | Composition of Besifloxacin and Isotretinoin Topical Gel (% w/w) | | | | | | | |
|---|---|---|---|---|---|---|---|---|
| Ingredients | TSG1 | TSG2 | TSG3 | TSG4 | TSG5 | TSG6 | TSG7 | TSG8 |
| Glycerin | 3.0 | 5.0 | 5.0 | 5.0 | 5.0 | 5.0 | 5.0 | 5.0 |
| Besifloxacin HCl | 1 | 1 | 0.5 | 2 | 1.5 | 0.5 | 4 | 0.5 |
| Purified water | q.s. | q.s. | q.s. | q.s. | q.s. | q.s. | q.s. | q.s. |
| | to 100 | to 100 | to 100 | to 100 | to 100 | to 100 | to 100 | to 100 |
| Edetate disodium | 0.1 | 0.1 | 0.1 | 0.1 | 0.1 | 0.1 | 0.1 | 0.1 |
| Hydroxyethyl | 1.2 | 1.2 | 1.2 | 0 | 1 | 0.8 | 0.6 | 1 |
| cellulose | | | | | | | | |
| Sodium | 0.2 | 0.2 | 0 | 0 | 0 | 0 | 0 | 0 |
| hyaluronate | | | | | | | | |
| Carbomer 980 | 0.1 | 0.5 | 0.85 | 0.5 | 0.85 | 0.5 | 0.85 | 0.85 |
| Phenoxyethanol | 0.7 | 0.7 | 0.7 | 0.7 | 0.7 | 0.7 | 0.7 | 0.7 |
| Isotretinoin | 0.05 | 0.075 | 0.1 | 0.7 | 0.05 | 0.05 | 0.025 | 0.05 |
| Tretinoin | 0 | 0 | 0 | 0 | 0 | 0 | 0 | 0.2 |
| Poloxamer 407 | 0.2 | 0.2 | 0.2 | 0 | 0 | 0 | 0 | 0 |
| Polyethylene | 0 | 5.0 | 7.0 | 10.0 | 2.0 | 5.0 | 8.0 | 2.0 |
| glycol 400 | | | | | | | | |
| Diethylene | 5.0 | 5.0 | 5.0 | 5.0 | 5.0 | 5.0 | 5.0 | 5.0 |
| glycol monoethyl | | | | | | | | |
| ether | | | | | | | | |
| Sod. Hydroxide | q.s. | q.s. | q.s. | q.s. | q.s. | q.s. | q.s. | q.s. |
| (18% w/w) | to pH | to pH | to pH | to pH | to pH | to pH | to pH | to pH |
| | 5.5-6.5 | 5.5-6.5 | 5.5-6.5 | 5.5-6.5 | 5.5-6.0 | 5.5-6.0 | 5.5-6.0 | 5.5-6.0 |

### Method of Preparation:

1. In a main mixing vessel, edetate disodium is dissolved in purified water under overhead stirring at 250 rpm.
2. Then vortex is created under overhead stirring at 300 rpm, and thickening agent hydroxyethyl cellulose is added slowly to avoid any lump formation. The contents of the beaker are stirred to obtain a lump free dispersion. This phase is named as hydroxyethyl cellulose phase.
3. In a separate vessel, carbomer is allowed to swell in purified water. The contents of the beaker are stirred for 30 min at 350 rpm using overhead stirrer till clear transparent phase is obtained. And neutralized with sodium hydroxide solution. This phase is named as Carbomer phase.
4. In a separate vessel purified water is loaded and sodium hyaluronate is added under stirring and allowed to swell for 1 hour. This phase is named as sodium hyaluronate phase.
5. In a vessel, glycerin is loaded. Besifloxacin hydrochloride is slowly added to it under stirring at 300 rpm. Then the dispersion is stirred, followed by addition of sodium hydroxide solution. The mixture is allowed to stir further for 15 minutes. Now, drug dispersion is ready to be transfer to main mixing vessel.
6. In another vessel (isotretinoin or tretinoin Phase), poloxamer is dissolved in purified water, then PEG 400, diethylene glycol monoethyl ether and isotretinoin or tretinoin is added one by one and mixed for 10 minutes.
7. Processing in main mixing vessel
   7.1. The content of carbomer phase is transferred to main mixing vessel containing hydroxyethyl cellulose phase and mixed for 20 minutes using overhead stirrer at 250 rpm.
   7.2. This is followed by addition of swelled sodium hyaluronate phase. The contents in the main mixing vessel are stirred for 5 minutes at 250 rpm using overhead stirrer.
   7.3. Then phenoxyethanol is directly added to the main mixing vessel and mixed for 30 minutes at 250 rpm using overhead stirrer.
   7.4. The besifloxacin phase is then added to the main mixing vessel and mixed.
   7.5. Isotretinoin phase is added to the main mixing vessel.
   7.6. pH of resulting gel is adjusted to 5.5-6.5 using sodium hydroxide solution.
   The contents in the main mixing vessel are stirred using overhead stirrer for additional 2 hr. A while to pale yellow homogenous gel is obtained.

### Example 15: Preparation of Besifloxacin HCl Loaded Gel Formulations with Isotonic Agent

Gel formulations containing suspended besifloxacin was prepared using isotonic agent such as sodium chloride as per the compositions shown in Table 1. The formulations had pH of 5.5-6.5 and other characterization details such as viscosity, assay of besifloxacin and phenoxyethanol are given below (Table 16).

TABLE 16
Gel Formulation Loaded with Suspended Besifloxacin
HCl for Composition NF1 using isotonic agent

| | Composition of Besifloxacin |
|---|---|
| | Topical Gel (% w/w) |
| Ingredients | NF1 |
| Glycerin | 5.0 |
| Besifloxacin HCl | 2.18 |
| Purified water | q.s to 100 |
| Edetate disodium | 0.1 |
| Hydroxyethyl cellulose | 0.60 |
| Sodium hyaluronate | 0.2 |
| Carbomer 980 | 0.65 |
| Phenoxyethanol | 0.7 |
| Polyethylene glycol 400 | 5.0 |
| Diethylene glycol monoethyl ether | 5.0 |
| Sodium Chloride | 0.9 |
| Sod. hydroxide solution | q.s. to pH 5.5-6.5 |

Characterization of Gel Formulations:

| Batch Code | NF1 |
|---|---|
| pH | 6.31 |
| Viscosity mPa · s* | 2597 |
| Assay of Besifloxacin | 1.96% w/w Label Claim (Assay: 98.67%) |
| Assay of Phenoxyethanol | 0.649% w/w Label Claim (Assay: 92.84%) |

*Viscosity was measured using Anton Paar viscometer

### Method of Preparation:

1. In a main mixing vessel, edetate disodium and sodium chloride were dissolved in purified water.
2. Then vortex was created under overhead stirring at 300 rpm, and thickening agent carbomer 980 was added slowly to avoid any lump formation. The contents of the beaker were stirred to obtain a lump free dispersion.
3. In a separate vessel, polyethylene glycol 400, diethylene glycol monoethyl ether and water were mixed. Then hydroxyethyl cellulose was added, and mixture was allowed to swell for 1 hour under stirring.
4. In a separate vessel, purified water was loaded, and sodium hyaluronate was added under stirring and allowed to swell for 1 hour.
5. In a vessel, glycerin was taken. Besifloxacin hydrochloride was slowly added to it and mixed. Then the dispersion was stirred, followed by addition of sodium hydroxide solution. The mixture was mixed for 5 minutes. Now, drug dispersion is ready for transfer to main mixing vessel.
6. Processing in main mixing vessel
   6.1. The content of hydroxyethyl cellulose phase was transferred to main mixing vessel containing carbomer phase and mixed for 10 minutes using overhead stirrer at 300 rpm.
   6.2. Step 6.1 was followed by addition of swelled sodium hyaluronate phase. The contents in the main mixing vessel are stirred for 10 minutes at 250 rpm using overhead stirrer.
   6.3. Then phenoxyethanol was added to the main mixing vessel and mixed for 20 minutes. This step was followed by addition of sodium hydroxide solution to adjust pH to 5.5-6.5.
   6.4. The besifloxacin phase was then added to the main mixing vessel and mixed.
   6.5. pH of resulting gel was adjusted to 5.5-6.5 using sodium hydroxide solution.
   The contents in the main mixing vessel are stirred using overhead stirrer for additional 2 hours to 3 hours. A white homogenous gel was obtained.

### Example 16: Preparation of Besifloxacin HCl Loaded Lotion with Isotonic Agent

Lotion containing suspended besifloxacin was prepared using isotonic agent such as sodium chloride, as per the compositions shown in Table 2. The formulations had a pH of 5.0-6.5 and other characterization details such as viscosity, assay of besifloxacin and phenoxyethanol are given below (Table 17).

TABLE 17
Lotion Formulations Loaded with Suspended Besifloxacin
for Compositions NF2 to NF3 using isotonic agent

| | Composition of Besifloxacin | |
|---|---|---|
| | Lotion (% w/w) | |
| Ingredients | NF2 | NF3 |
| Glycerin | 5.0 | 5.0 |
| Besifloxacin HCl | 2.18 | 1.09 |
| Purified water | q.s to 100 | q.s to 100 |
| Edetate disodium | 0.1 | 0.1 |
| Hydroxyethyl cellulose | 0.17 | 0 |
| Sodium hyaluronate | 0.2 | 0.2 |
| Carbomer 980 | 1.05 | 1.05 |
| Phenoxyethanol | 0.7 | 0.7 |
| Polyethylene glycol 400 | 5.0 | 3.0 |
| Diethylene glycol monoethyl ether | 5.0 | 4.0 |
| Sodium Chloride | 0.9 | 0.9 |
| Sod. hydroxide solution | q.s. to pH 5.0-6.5 | q.s. to pH 5.0-6.5 |

Characterization of Formulations:

| Batch Code | NF2 | NF3 |
|---|---|---|
| pH | 5.10 | 6.2 |
| Viscosity mPa · s* | 1525 | 4381 |
| Assay of Besifloxacin | 2.01% w/w Label Claim | Not Determined |
| | (Assay: 100.26%) | |
| Assay of Phenoxyethanol | 0.69% w/w Label Claim | Not Determined |
| | (Assay: 99.14%) | |

*Viscosity was measured using Anton Paar viscometer

### Method of Preparation:

1. In a main mixing vessel, edetate disodium and sodium chloride were dissolved in purified water.
2. Then vortex was created under overhead stirring at 300 rpm, and thickening agent carbomer 980 was added slowly to avoid any lump formation. The contents of the beaker are stirred to obtain a lump free dispersion.
3. In a separate vessel, polyethylene glycol 400, diethylene glycol monoethyl ether and water were mixed. Then hydroxyethyl cellulose was added and allowed to swell for 1 hour using overhead stirrer.
4. In a separate vessel, purified water was loaded and sodium hyaluronate was added to water under stirring and allowed to swell for 1 hour.
5. In a vessel, glycerin was loaded. Besifloxacin hydrochloride was slowly added to it and mixed. Then the dispersion is stirred, followed by addition of sodium hydroxide solution. The mixture was allowed to stir further for 5 minutes. Now, drug dispersion was ready to be transfer to main mixing vessel.
6. Processing in main mixing vessel
   6.1. The content of hydroxyethyl cellulose phase was transferred to main mixing vessel containing carbomer phase and mixed for 10 minutes using overhead stirrer at 300 rpm.
   6.2. This was followed by addition of swelled sodium hyaluronate phase. The contents in the main mixing vessel are stirred for 10 minutes at 250 rpm using overhead stirrer.
   6.3. Then phenoxyethanol was directly added to the main mixing vessel and mixed for 20 minutes at 250 rpm. This step was followed by addition of sodium hydroxide solution to adjust pH to 5.0-6.5.
   6.4. The besifloxacin phase was then added to the main mixing vessel and mixed.
   6.5. pH of resulting lotion was adjusted to 5.0-6.5 using sodium hydroxide solution.
   6.6. The contents in the main mixing vessel are stirred using overhead stirrer for additional 2 hour to 3 hours. A white homogenous lotion was obtained.

### Example 17: Preparation of Besifloxacin HCl Lotion with Homogenization of Drug Phase

Lotion containing suspended besifloxacin was prepared using homogenization of drug phase, as per the compositions shown in Table 3. The particle size of active phase was found to be 2.3 μm with PDI:0.326. The final formulations had a pH of 5.1 and other characterization details such as viscosity, assay of besifloxacin and phenoxyethanol are given below (Table 18).

TABLE 18
Lotion Formulations Loaded with Suspended
Besifloxacin•HCl for Compositions NF4

| | Composition of Besifloxacin |
|---|---|
| | Lotion (% w/w) |
| Ingredients | NF4 |
| Glycerin | 5.0 |
| Besifloxacin HCl | 2.18 |
| Purified water | q.s to 100 |
| Edetate disodium | 0.1 |
| Hydroxyethyl cellulose | 0.17 |
| Sodium hyaluronate | 0.2 |
| Carbomer 980 | 1.05 |
| Phenoxyethanol | 0.7 |
| Polyethylene glycol 400 | 5.0 |
| Diethylene glycol monoethyl ether | 5.0 |
| Sodium Chloride | 0.9 |
| Sod. hydroxide solution | q.s. to pH 5.0-6.5 |

Characterization of Formulations:

| Batch Code | NF4 |
|---|---|
| pH | 5.12 |
| Viscosity mPa · s* | 1422 |
| Assay of Besifloxacin | 1.96% w/w Label Claim (Assay: 97.89%) |
| Assay of Phenoxyethanol | 0.68% w/w Label Claim (Assay: 96.98%) |

### Method of Preparation:

1. The manufacturing procedure of lotion formulation was same as of example no. 16 given above, except for homogenization of besifloxacin dispersion phase to obtain particle size less than 10 μm.

### Example 18: Preparation of Besifloxacin HCl Loaded Ointment

Besifloxacin ointment containing suspended besifloxacin was prepared as per the compositions shown in Table 4. The formulations had a pH of 5.5-6.5 and other characterization details such as viscosity, assay of besifloxacin are given below (Table 19).

TABLE 19
Ointment Formulations Loaded with Suspended Besifloxacin•HCl
for Compositions NF5, NF6, NF8 and NF9

Composition of Besifloxacin
Ointment (% w/w)

| Ingredients | NF5 | NF6 | NF8 | NF9 |
|---|---|---|---|---|
| Glycerin | 5.0 | 5.0 | 5.0 | 0 |
| Besifloxacin HCl | 2.18 | 2.18 | 2.18 | 1.09 |
| Polyethylene glycol 4000 | 30.0 | 30.0 | 0 | 0 |
| Polyethylene glycol 400 | 40.0 | 40.0 | 0 | 0 |
| Propylene glycol | 22.87 | 16.98 | 0 | 0 |
| Sodium Chloride | 0 | 0.9 | 0 | 0 |
| Purified Water | 0 | 5.0 | 0 | 0 |
| Mineral oil | 0 | 0 | 5 | 10 |
| White soft paraffin | 0 | 0 | q.s. | q.s. |
| | | | to 100 | to 100 |

Characterization of Formulations:

| Batch Code | NF5 | NF6 | NF8 | NF9 |
|---|---|---|---|---|
| pH | 5.97 | 6.23 | — | — |
| Viscosity mPa · s* | 24620 | 29370 | — | — |
| Assay of Besifloxacin | Not | 1.93% w/w | — | — |
| | Determined | Label Claim | | |
| | | (Assay: 96.46%) | | |

### Method of Preparation:

1. In main mixing vessel, polyethylene glycol 400 and polyethylene glycol 4000, or white soft paraffin were heated to 75° C.±5° C. in mixer to melt. Mixture was cooled down to 45° C. with mixing.
2. In a side vessel, besifloxacin was dispersed in glycerin. The drug dispersion was transferred to mixer at step-1.
3. The drug dispersion container was rinsed with propylene glycol or mineral oil and the washings were added to main mixing vessel.
4. Mixed at high speed at 45°±2° C. for 15 minutes.
5. Aqueous sodium chloride solution was added to step 4 for ointment compositions detailed in table 19 and mixed.
6. Obtained ointment temperature was cooled down to 25° to 30° C. with continuous mixing for 2 hours.

### Example 19: Preparation of Besifloxacin HCl Loaded Spray

Besifloxacin spray containing suspended besifloxacin was prepared as per the composition shown in Table 5. The formulations had pH of 5.0-6.5 and other characterization details such as viscosity, is given below (Table 20).

TABLE 20
Spray Formulations Loaded with Suspended
Besifloxacin•HCl for Composition NF7

| | Composition of Besifloxacin |
|---|---|
| | Spray (% w/w) |
| Ingredients | NF7 |
| Glycerin | 2.5 |
| Besifloxacin HCl | 1.0 |
| Sodium Carboxy Methyl cellulose | 1.5 |
| Phenoxyethanol | 0.7 |
| Sod. hydroxide | q.s. to pH 5.0-6.5 |
| Purified Water | q.s. to 100 |

Characterization of Formulations:

| Batch Code | NF7 |
|---|---|
| pH | 5.5 |
| Viscosity mPa · s* | 105.7 |

*Viscosity was measured using Anton Paar viscometer

### Method of Preparation:

1. In main mixing vessel, sodium carboxymethyl cellulose was swelled in purified water.
2. Phenoxyethanol was added to above container and mixed.
3. In side vessel, besifloxacin was dispersed in glycerin. Then pH of dispersion was adjusted to 5.0-6.5 by addition of sodium hydroxide solution.
4. Transferred the drug dispersion to step-2 and mixed for 60 minutes.
5. Spray was obtained.

### Example 20: Preparation of Formulations Containing Besifloxacin and its Combination with Adapalene

Formulations comprising besifloxacin in combination with adapalene were prepared. Tables 21 to 26 below provide the said formulations:

TABLE 21
Adapalene GL 1

| | | |
|---|---|---|
| 1 | Water | q.s |
| 2 | Carbopol 940 | 1 |
| 3 | Allantoin | 0.2 |
| 4 | Besifloxacin HCl | 1 (D1) |
| | (equivalent to | |
| | besifloxacin) | |
| 5 | Adapalene | 0.1 |
| 6 | Triethanolamine | 1 |
| 7 | Glycerol | 5 |
| 8 | Propylene Glycol | 5 |
| 9 | PEG 400 | 5 |
| 10 | Poloxamer 407 | 0.2 |
| 11 | Disodium EDTA | 0.1 |
| 12 | Phenoxyethanol | 0.5 |

TABLE 22
Adapalene S1

| | | |
|---|---|---|
| 1 | Water | q.s |
| 2 | Sodium hydroxide (18% aq.) | q.s |
| 3 | PEG 1450 | 2 |
| 4 | Methyl Gluceth-20 | 2.5 |
| 5 | Glycerin | 1 |
| 6 | Besifloxacin | 1 |
| 7 | Adapalene | 0.1 |
| 8 | Isopropyl alcohol | 20 |
| 9 | diethylene glycol monethyl ether | 1 |
| 10 | Propylene glycol | 1.5 |
| 11 | N-methyl 2-pyrrolidone | 3 |
| 12 | Sodium hydroxide | q.s |
| 13 | Phenoxyethanol | 1 |
| 14 | Fragrance | 0.4 |

TABLE 23
Adapalene FW1

| | | |
|---|---|---|
| 1 | Water | q.s. |
| 2 | Carbopol aqua SF-1 | 6 |
| 3 | Sodium C14-16 Olefin Sulfonate | 35 |
| | Sulfonate | |
| 4 | Sodium lauryl ether sulphate | 2 |
| 5 | Sodium hydroxide (18% aq.) | q.s. |
| 6 | Cocamidopropylbetaine (30%) | 10 |
| 7 | Disodium EDTA | 0.1 |
| 8 | Glycerin | 5 |
| 9 | Besifloxacin | 1 |
| 10 | Adapalene | 0.1 |
| 11 | N-methyl 2-pyrrolidone | 3 |
| 12 | PEG-7 glycerylcocoate | 1 |
| 13 | Citric Acid (50%) | q.s. |

TABLE 24
Adapalene SB1

| | | |
|---|---|---|
| 1 | Sodium palmitate | 94.2 |
| 2 | Sodium lauryl ether sulphate | 2 |
| 3 | Polyquatemium-39 | 1 |
| 4 | Methyl Gluceth-20 | 1 |
| 5 | Titanium dioxide | 0.5 |
| 6 | Besifloxacin | 1 |
| 7 | Adapalene | 0.1 |
| 8 | Oleyl oleate | 0.5 |
| 9 | BHT (Butylated Hydroxy Toluene) | 0.01 |

TABLE 25
Adapalene BW1

| | | |
|---|---|---|
| 1 | Water | q.s. |
| 2 | Disodium EDTA | 0.1 |
| 3 | Carbopol aqua SF-1 | 1 |
| 4 | Ammonium lauryl sulphate (30%_{—} | 30 |
| 5 | Propylene glycol | 5 |
| 6 | Besifloxacin | 1 |
| 7 | Adapalene | 0.1 |
| 8 | N-methyl 2-pyrrolidone | 3 |
| 9 | Ethanol | 4 |
| 10 | Propyl paraben | 0.03 |
| 11 | Methyl gluceth-10 | 0.3 |
| 12 | Disodium laureth sulfosuccinate (39%) | 2 |
| 13 | Fragrance | 0.5 |
| 14 | Triethanolamine | q.s. |

TABLE 26
Adapalene L1

| | | |
|---|---|---|
| 1 | Water | q.s |
| 2 | Disodium EDTA | 0.05 |
| 3 | Carbopol aqua SF-1 | 1 |
| 4 | Petrolatum | 1 |
| 5 | Cyclomethicone | 0.5 |
| 6 | Sorbitan stearate | 1.4 |
| 7 | Polysorbate 60 | 0.6 |
| 8 | Methyl gluceth-20 | 1 |
| 9 | Cetyl alcohol | 1.6 |
| 10 | Tocopheryl acetate | 0.25 |
| 11 | Besifloxacin | 1 |
| 12 | Adapalene | 0.1 |
| 13 | N-methyl 2-pyrrolidone | 3 |
| 14 | Propylene glycol | 2 |
| 15 | Glycerin | 8 |
| 16 | Ethanol | 2 |
| 17 | Phenoxyethanol | 1 |
| 18 | Sodium hydroxide | q.s |

TABLE 27
Adapalene SL1

| | | |
|---|---|---|
| 1 | Besifloxacin•HCl Equivalent | 1 |
| | to Besifloxacin | |
| 2 | Adapalene | 0.1 |
| 3 | Allantoin | 0.2 |
| 4 | Diethylene glycol monoethyl ether | 11 |
| 5 | Edetate disodium dihydrate | 0.1 |
| 6 | Glycerin | 5 |
| 7 | Hyaluronate Sodium | 0.3 |
| 8 | Hydroxy ethylcellulose | 0.9 |
| 9 | Phenoxyethanol | 0.7 |
| 10 | Poloxamer | 0.2 |
| 11 | Polyethylene glycol 400 | 6 |
| 12 | Purified water | q.s. to 100 |

TABLE 28
Adapalene GA5

| | | |
|---|---|---|
| 1 | Adapalene | 0.1 |
| 2 | Besifloxacin•HCl equivalent | 1 |
| | to besifloxacin | |
| 3 | Allantoin | 0.2 |
| 4 | Citric acid solution | 0.15 |
| 5 | Diethylene glycol monoethyl ether | 5 |
| 6 | Edetate disodium dehydrate | 0.1 |
| 7 | Glycerin | 5 |
| 8 | Hyaluronate Sodium | 0.2 |
| 9 | Hydroxy ethyl cellulose | 1.2 |
| 10 | Phenoxyethanol | 0.7 |
| 11 | Poloxamer 407 | 0.2 |
| 12 | Polyethylene glycol 400 | 5 |
| 13 | Sodium Hydroxide Solution | q.s. |
| 14 | Purified Water | q.s. |

TABLE 29
Adapalene GL19

| | | |
|---|---|---|
| 1 | Besifloxacin•HCl equivalent | 1 |
| | to besifloxacin | |
| 2 | Adapalene | 0.1 |
| 3 | Allantoin | 0.2 |
| 4 | Carbomer homopolymer type C | 0.85 |
| 5 | Diethylene glycol monoethyl ether | 5 |
| 6 | Edetate disodium dehydrate | 0.1 |
| 7 | Glycerin | 5 |
| 8 | Hyaluronate sodium | 0.4 |
| 9 | Phenoxyethanol | 0.7 |
| 10 | Poloxamer 407 | 0.2 |
| 11 | Polyethylene glycol 400 | 5 |
| 12 | Sodium hydroxide solution | q.s. |
| 13 | Purified water | q.s. to 100 |

### Example 21: In-Vitro Release Testing of Besifloxacin Topical Gel 1%

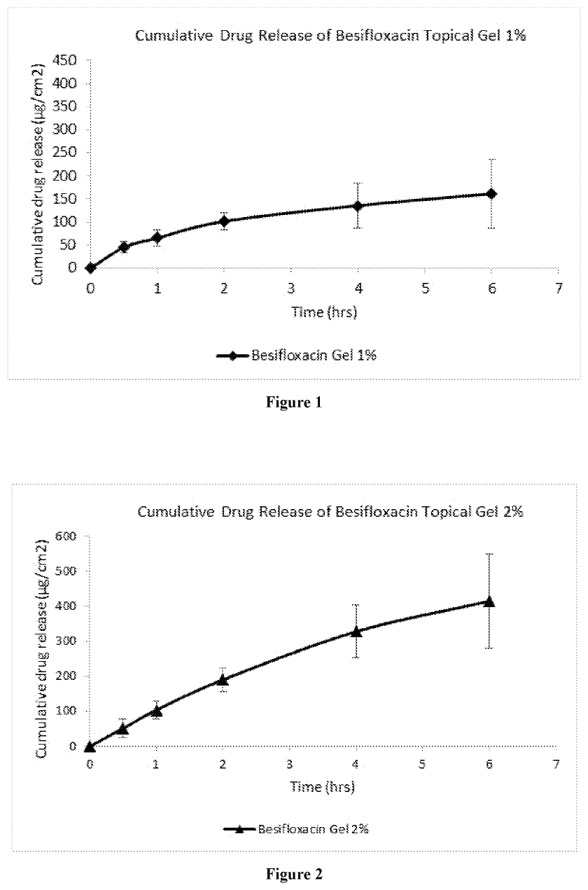

Gel formulations containing suspended besifloxacin were tested for in-vitro release testing. Briefly, franz diffusion cell assembly was used for in-vitro release testing of besifloxacin formulations. Receptor compartment was filled with citrate buffer pH 4.0 and maintain temperature at 32° C.±1° C. while stirring at 700 rpm. The Polysulfone membrane were fixed between donor and receptor compartment of Franz diffusion cell. The set assembly was left completely undisturbed for 30 min to equilibrate membrane. Then the stirring of the instrument was turned off just before gel application to the membrane. The besifloxacin gel was evenly applied over membrane with spatula to the exposed area fixed on Franz diffusion cell. Then stirring of the Franz diffusion cell was turned on and recorded initial time point. Release of besifloxacin from donor compartment to receptor compartment was monitored at predetermined time points of 0.5, 1, 2, 4 and 6 h. Turned off stirring before sampling. At defined time points, release medium aliquot (1 ml) of the receptor compartment was collected and replaced with fresh release medium, so that the lower surface of the membrane remains in contact with the receptor compartments over the experimental time period. At the end, release profile of besifloxacin from formulation was determined, and observed as in FIG. 1.

### Example 22: In-Vitro Release Testing of Besifloxacin Topical Gel 2%

Besifloxacin topical gel (2%) containing suspended besifloxacin were tested for in-vitro release testing. Release testing procedure was similar to example 21. Release profile is given in FIG. 2.

### Example 23: In-Vitro Release Testing of Besifloxacin Ointment 1%

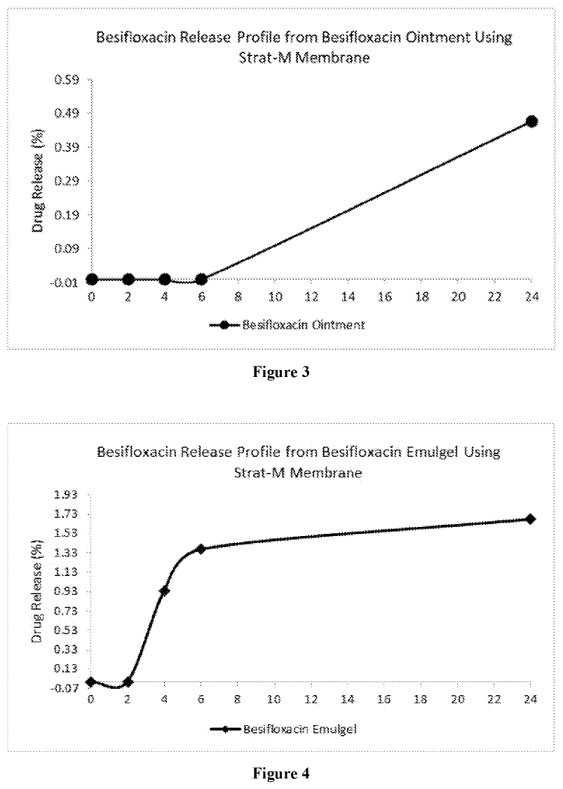

Besifloxacin ointment formulations containing suspended besifloxacin were tested for in-vitro release testing. Release testing procedure was similar to example 21, where release medium, membrane used for study were ethanolic water (20.0%) and Start-M®, respectively. Release profile is given in FIG. 3.

### Example 24: In-Vitro Release Testing of Besifloxacin Emulgel 1%

Besifloxacin emulgel formulations containing suspended besifloxacin were tested for in-vitro release testing. Release testing procedure was similar to example 21, where release medium, dose of besifloxacin formulation applied and membrane used for study were ethanolic water (20.0%), 25 mg/cm2 and Start-M®, respectively. Release profile is given below in FIG. 4.

### Example 25: Skin Retention of Besifloxacin Cream on Pig Ear Skin

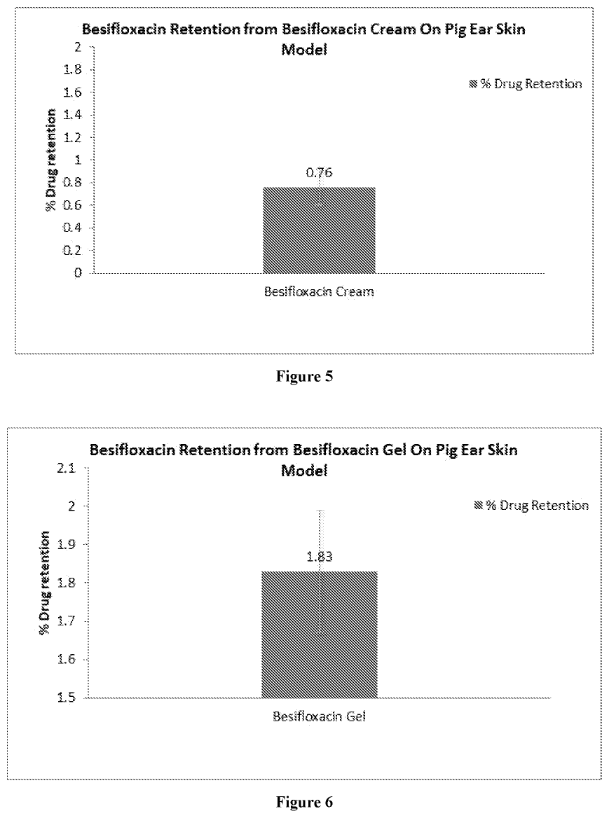

Besifloxacin cream containing suspended besifloxacin was tested for skin retention study on pig ear skin. Franz diffusion cell assembly was used for ex-vivo skin retention study. The receptor compartment was filled with Phosphate buffer saline with 0.005% albumin, pH 7.4 and maintain temperature at 32° C.±1° C. while stirring at 700 rpm. The processed pig ear skin was equilibrated with receptor medium for 30 minutes and then fixed between donor and receptor compartment of Franz diffusion cell. Then the stirring of the instrument was turned off just before gel application to the membrane. The besifloxacin gel was evenly applied over skin with spatula to the exposed area fixed on Franz diffusion cell. Then stirring of the Franz diffusion cell was turned on and recorded initial time point. At the end of study (5 hrs to 6 hrs), the excess formulation remaining on the surface of pig ear skin was removed with the help of 3 cotton balls (weight of each cotton ball=0.5 g). Drug was extracted from pig ear skin and the extract samples were submitted for HPLC analysis. HPLC results of release samples, skin extracts and cotton ball extract were used for calculation of release profile, skin retention and recovery of drug from cotton ball, respectively. Skin retention results are shown in FIG. 5.

### Example 26: Skin Retention of Besifloxacin Gel on Pig Ear Skin

Besifloxacin gel containing suspended besifloxacin was tested for skin retention study on pig ear skin. Testing procedure was similar to example 26. Skin retention results are shown in FIG. 6.

### Example 27: Skin Penetration of Besifloxacin Cream on Human Skin

Besifloxacin gel and cream containing suspended besifloxacin were tested for skin retention study on human abdominal skin. Besifloxacin gel and cream were applied over a 12 hr application period. Heat-separated epidermal membranes from a single female donor were mounted in horizontal Franz-type diffusion cells (exposed surface area 1.33 cm2) over a receptor solution of phosphate buffered saline, pH 7.4 containing 5% ethanol to facilitate solubilisation of any material penetrating the skin. Cells were maintained at 35° C. and receptor solution was stirred throughout. Formulations containing approximately 1.0% besifloxacin w/w and applied at a dose of approximately 10 mg/cm². The samples of the receptor solution were taken at times up to 12 hrs and extracts from surface swabs, stratum corneum tape strips and residual epidermis were obtained at the end of the experiment at 12 hrs. All samples were analysed by validated LCMS.

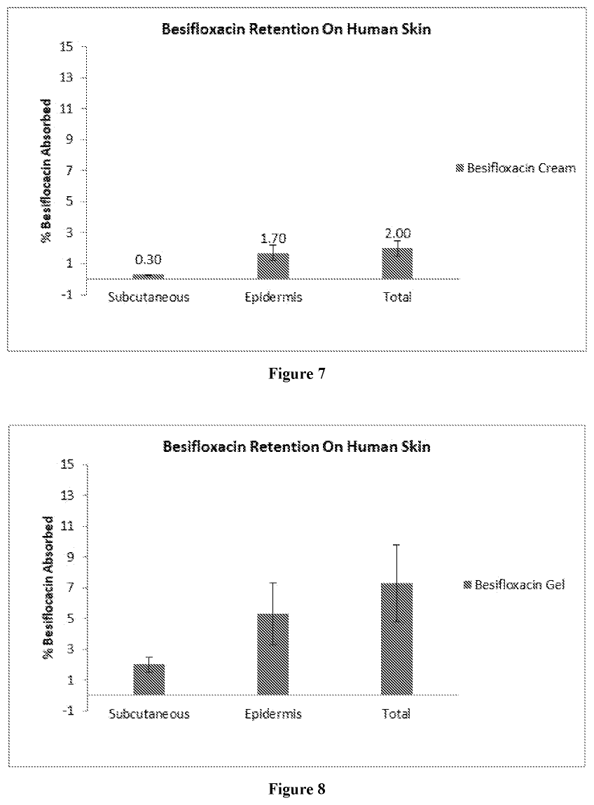

The results suggest that negligible amounts of besifloxacin were recovered from receptor solution with formulation. However, after application of cream, significantly greater amounts of besifloxacin were recovered from the viable epidermis and the entire epidermal membrane (Subcutaneous+epidermis) (FIG. 7).

### Example 28: Skin Penetration of Besifloxacin Gel on Human Skin

Besifloxacin gel containing suspended besifloxacin was tested for skin retention study on human abdominal skin. Testing procedure was similar to example 27. Skin retention results are shown in FIG. 8.

Drug release is an important property of a therapeutic agent and is a prerequisite for its absorption and penetration of drug to the site of action on the skin layer. Demonstration of drug release from a formulation attests to its availability on the hair follicles and upper layer of skin where pathogen responsible for disease resides, thus resulting in its activity against the target disease/condition. The above generated data suggests that amount of drug released and retained onto the skin should be sufficient to exert its therapeutic activity.

### Example 29: Activity of Besifloxacin Gel and Ointment Wild Type and Multi-Drug Resistant E. coli

Besifloxacin ointment (2%) and gel (2%) formulations were tested on E. coli MTCC 1687 (wild type strain) and E. coli ATCC BAA196 (multi-drug resistant strain), and the results are as provided in table 30 below:

TABLE 30

| | | E. coli MTCC1687 | | E. coli BAA196 | |
|---|---|---|---|---|---|
| S. | | weight* | ZOI | weight | ZOI |
| No | Formulations | (mg) | (mm) | (mg) | (mm) |
| 1 | Besifloxacin | 4.8 | 28 | 5.0 | 26 |
| | 2% Ointment | 3.9 | 24 | 5.1 | 26 |
| | (VLNF/55/PGK/010 | 5.5 | 26 | 5.2 | 23 |
| | Average | 4.7 | 26 | 5.1 | 25 |
| 2 | Besifloxacin | 6.4 | 23 | 6.3 | 22 |
| | 2% Gel | 4.3 | 21 | 6.5 | 21 |
| | (PT6054) | 6.1 | 20 | 5.3 | 18 |
| | Average | 5.6 | 21 | 6.0 | 20 |

*Actual weight of the formulation loaded on the disc to perform ZOI. (MTTC 1687- Std. Strain & MTCC BAA 196- multi drug resistant strain)

As seen, both the formulations showed similar ZOI value against susceptible and multi drug resistant E. coli strains, confirming that the formulations of the present disclosure are effective against gram negative organisms.

All patents and other publications identified in the specification and examples are expressly incorporated herein by reference for all purposes. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow. Further, to the extent not already indicated, it will be understood by those of ordinary skill in the art that any one of the various embodiments herein described and illustrated can be further modified to incorporate features shown in any of the other embodiments disclosed herein.

Additional embodiments and features of the present disclosure will be apparent to one of ordinary skill in art based on the description provided herein. The embodiments herein provide various features and advantageous details thereof in the description. Descriptions of well-known/conventional methods and techniques are omitted so as to not unnecessarily obscure the embodiments herein.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments in this disclosure have been described in terms of preferred embodiments, those skilled in the art will recognize that the embodiments herein can be practiced with modification within the spirit and scope of the embodiments as described herein.

Any discussion of documents, acts, materials, devices, articles and the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

While considerable emphasis has been placed herein on the particular features of this disclosure, it will be appreciated that various modifications can be made, and that many changes can be made in the preferred embodiments without departing from the principles of the disclosure. These and other modifications in the nature of the disclosure or the preferred embodiments will be apparent to those skilled in the art from the disclosure herein, whereby it is to be distinctly understood that the foregoing descriptive matter is to be interpreted merely as illustrative of the disclosure and not as a limitation.

### REFERENCES

1. Neubert, U., Jansen, T. and Plewig, G. (1999), Bacteriologic and immunologic aspects of Gram-negative folliculitis: a study of 46 patients. International Journal of Dermatology, 38: 270-274.
2. Leyden, J. J., Marples, R. R., Mills, O. H. and Kligman, A. M. (1973), Gram-negative folliculitis—a complication of antibiotic therapy in acne vulgaris. British Journal of Dermatology, 88: 533-538.

1. A method for treating gram-negative folliculitis in a subject, said method comprising topically administering a therapeutically effective amount of a formulation of besifloxacin at a concentration ranging from about 0.5% to about 4%.

2. The method of claim 1, wherein the formulation of besifloxacin is gel, cream, lotion, foam, emulgel, ointment or spray.

3. The method of claim 2, wherein the formulation of besifloxacin is an aqueous formulation having a pH ranging from about 5 to about 8, or is a non-aqueous pH independent formulation.

4. The method of claim 2, wherein the formulation, in addition to besifloxacin, comprises excipients selected from a group comprising anti-acne agent, alkalizing agent, anti-oxidant, anti-microbial agent, chelating agent, conditioning agent, dispersing agent, emollient, emulsifier, humectant, moisturizer, isotonic agent, foam stabilizer, solubilizer, thickening agent, penetration enhancer, preservative, solvent, surfactant, stabilizer, lubricant, opacifier and viscosity modifier.

5. The method of claim 4, wherein the alkalizing agent is selected from a group comprising sodium hydroxide and triethanolamine or a combination thereof; the anti-oxidant is selected from a group comprising butylated hydroxytoluene (BHT) and D-α-tocopherol polyethylene glycol succinate (TPGS) or a combination thereof; the anti-microbial agent is phenonip; the chelating agent is selected from a group edetate disodium and edetate disodium dihydrate or a combination thereof; the conditioning agent is cyclopentasiloxane; the dispersing agent is selected from a group comprising poloxamer 407 and poloxamer 124 or a combination thereof; the emollient is selected from a group comprising behenyl alcohol, cyclomethicone, oleyl oleate, and light liquid paraffin or a combination thereof; the emulsifier is selected from a group comprising Brij 35, cetyl alcohol, glyceryl stearate, glyceryl monostearate, laureth 4, PEG-400 stearate, polysorbate 60, steareth 2, sodium palmitate and steareth 21 or any combination thereof; the humectant is selected from a group comprising glycerine, methyl gluceth-20, and propylene glycol or a combination thereof; the moisturizer is allantoin; the isotonic agent is sodium chloride; the foam stabilizer is cocamidopropylbetaine; the solubilizer is selected from a group comprising caproyl 90, diethylene glycol monoethyl ether, N-methyl 2-pyrrolidone and polyethylene glycol 400 or any combination thereof; the thickening agent is selected from a group comprising carbomer homopolymer type C, carbomer, carbopol 980, hydroxyethyl cellulose, pemulen, sepineo P600, sodium hyaluronate, stearyl alcohol, ultrez 21 and xanthan gum or any combination thereof; the preservative is selected from a group comprising phenoxyethanol and propyl paraben; the solvent is purified water; the lubricant is PEG-7 glycerylcocoate; the opacifier is titanium dioxide; the viscosity modifier is selected from a group comprising carbopolaqua SF-1 and petrolatum; and the surfactant is selected from a group comprising sodium lauryl sulphate, sodium C14-16 olefin sulfonate, sodium lauryl ether sulphate, polyquaternium-39, ammonium lauryl sulphate (30%), disodium laureth sulfosuccinate (39%), sorbitan stearate and tween 80 or a combination thereof.

6. The method of claim 5, wherein the sodium hydroxide is at a concentration ranging from about 0.04% to about 1.2%, the triethanolamine is at a concentration of about 1%, the butylated hydroxytoluene (BHT) is at a concentration of about 0.1%, the D-α-tocopherol polyethylene glycol succinate (TPGS) is at a concentration ranging from about 3% to about 5%, the phenonip is at a concentration ranging from about 0.3% to about 0.4%, the edetate disodium or the edetate disodium dihydrate is at a concentration of about 0.1%, the cyclopentasiloxane is at a concentration of about 5%, the poloxamer 407 or the poloxamer 124 is at a concentration ranging from about 0.5% to about 1%, the behenyl alcohol is at a concentration ranging from about 1% to about 1.5%, the cyclomethicone is at a concentration ranging from about 1% to about 6%, the oleyl oleate is at a concentration of about 0.5%, the light liquid paraffin is at a concentration ranging from about 2% to about 7%, the Brij 35 is at a concentration of about 5.1%, the cetyl alcohol is at a concentration ranging from about 1% to about 2%, the glyceryl stearate or the glyceryl monostearate is at a concentration ranging from about 1.5% to about 3%, the laureth 4 is at a concentration of about 4%, the PEG-400 stearate is at a concentration ranging from about 2% to about 10%, the polysorbate 60 is at a concentration ranging from about 2% to about 4%, the sodium palmitate is at a concentration of about 94.2%, the steareth 2 or the steareth 21 is at a concentration ranging from about 2% to about 3%, the glycerin is at a concentration ranging from about 1% to about 10%, the Methyl Gluceth-20 is at a concentration ranging of about 0.3% to about 2.5%, the propylene glycol is at a concentration ranging from about 1% to about 22%, the allantoin is at a concentration of about 0.2%, the sodium chloride is at a concentration of about 0.9%, the cocamidopropylbetaine is at a concentration of about 0.5%, the caproyl 90 is at a concentration ranging from about 4% to about 5%, the diethylene glycol monoethyl ether is at a concentration ranging from about 1% to about 16%, the N-methyl 2-pyrrolidone is at a concentration of about 3%, the polyethylene glycol 400 is at a concentration ranging from about 0.1% to about 8%, the carbomer homopolymer type C is at a concentration ranging from about 0.3% to about 0.65%, the carbomer or the carbopol 980 is at a concentration ranging from about 0.1% to about 25%, the hydroxyethyl cellulose is at a concentration ranging from about 0.17% to about 1.75%, the pemulen is at a concentration ranging from about 1% to about 40%, the sepineo P600 is at a concentration ranging from about 4% to about 5%, the sodium hyaluronate is at a concentration ranging from about 0.1% to about 0.5%, the stearyl alcohol is at a concentration ranging from about 1% to about 2%, the ultrez 21 is at a concentration ranging from about 5% to about 20%, the xanthan gum is at a concentration ranging from about 0.5% to about 0.6%, the phenoxyethanol is at a concentration of about 0.5%, the propyl paraben is at a concentration of about 0.03%, the PEG-7 glycerylcocoate is at a concentration of about 1%, the titanium dioxide is at a concentration of about 0.5%, the carbopolaqua SF-1 is at a concentration ranging from about 1% to about 6%, the petrolatum is at a concentration of about 1%, the sodium lauryl sulphate is at a concentration of about 5%, the sodium C14-16 olefin sulfonate is at a concentration of about 35%, the sodium lauryl ether sulphate is at a concentration of about 2%, the polyquaternium-39 is at a concentration of about 1%, the ammonium lauryl sulphate (30%) is at a concentration of about 30%, the disodium laureth sulfosuccinate (39%) is at a concentration of about 2%, the sorbitan stearate is at a concentration of about 1.4% and the tween 80 is at a concentration of about 8%.

7. The method of claim 1, wherein the gram-negative folliculitis is caused by gram-negative bacteria selected from a group comprising Escherichia coli, Klebsiella spp., Pseudomonas spp., Serratia spp., Acinetobacter spp., Enterobacter spp. and Proteus spp.

8. The method of claim 7, wherein the gram-negative folliculitis is caused by gram-negative bacteria selected from a group comprising Klebsiella pneumoniae, Pseudomonas aeruginosa, Acinetobacter baumannii, Enterobacter aerogenes and Proteus mirabilis.

9. The method of claim 8, wherein the besifloxacin inhibits the gram-negative bacteria, and wherein minimum inhibitory concentration (MIC) of the besifloxacin against the gram-negative bacteria is lower than the MIC of antibiotics selected from a group comprising cefotaxime and ampicillin.

10. The method of claim 1, wherein the gram-negative folliculitis is caused by gram-negative bacteria which is resistant to conventional antibiotics and fluoroquinolone other than besifloxacin.

11. The method of claim 10, wherein the gram-negative bacteria is resistant to ampicillin, amoxicillin, cefotaxime, clindamycin, tetracycline or erythromycin.

12. (canceled)

13. (canceled)

14. The method of claim 1, wherein the topical administration of the besifloxacin is carried out at least once a day to up to four times a day; and wherein each administration is in an amount ranging from about 2 finger-tip unit (FTU) to about 4.5 finger-tip unit (FTU), or from about 1 gram to about 2.5 grams.

15. (canceled)

16. The method of claim 1, wherein the formulation, in addition to besifloxacin, comprises a second active agent selected from a group comprising retinoid derivative, sebum inhibitor, antibiotic and anti-inflammatory agent, or any combination thereof.

17. (canceled)

18. The method of claim 16, wherein the second active agent is at a concentration ranging from about 0.001% to about 10%.

19. (canceled)

20. A formulation for treating gram-negative folliculitis or inflammation associated with gram-negative folliculitis in a subject, comprising besifloxacin at a concentration ranging from about 0.5% to about 4%.

21. The formulation of claim 20, wherein the formulation is selected from a group comprising gel, cream, lotion, foam, emulgel, ointment and spray; and in addition to the besifloxacin comprises excipients selected from a group comprising anti-acne agent, alkalizing agent, anti-oxidant, anti-microbial agent, chelating agent, conditioning agent, dispersing agent, emollient, emulsifier, humectant, moisturizer, isotonic agent, foam stabilizer, solubilizer, thickening agent, penetration enhancer, preservative, solvent, surfactant, stabilizer, lubricant, opacifier and viscosity modifier

22. The formulation of claim 20, comprising: about 0.5 to about 4 (% w/w) besifioxacin.HCl (Equivalent to Besifloxacin); about 2 to about 7 (% w/w) diethylene glycol monoethyl ether; about 0.1 (% w/w) edetate disodium dihydrate (EDTA); about 2 to about 10 (% w/w) glycerin; about 0.9 to about 1.75 (% w/w) hydroxyethyl cellulose; 0 to about 0.8 (% w/w) carbomer; about 0.3 to about 0.7 (% w/w) phenoxyethanol; about 2 to about 7 (% w/w) polyethylene glycol 400; 0 to about 0.5 (% w/w) sodium hyaluronate; sodium hydroxide; and purified water.

23. The formulation of claim 20, comprising: about 1 to about 4 (% w/w) besifioxacin.HCl (equivalent to besifloxacin); about 5 (% w/w) diethylene glycol mono ethyl ether; about 0.1 (% w/w) edetate disodium dihydrate (EDTA); about 5 (% w/w) glycerin; about 0.5 to about 1.5 (% w/w) hydroxyethyl cellulose; about 0.3 to about 1.2 (% w/w) carbomer; about 0.7 (% w/w) phenoxyethanol; about 5 (% w/w) polyethylene glycol 400; 0 to about 1 (% w/w) sodium hyaluronate; sodium hydroxide; and purified water.

24. The formulation of claim 20, wherein the formulation, in addition to besifloxacin, comprises a second active agent selected from a group comprising retinoid derivative, sebum inhibitor, antibiotic and anti-inflammatory agent, or any combination thereof.

25. (canceled)

26. The formulation of claim 24, wherein the second active agent is at a concentration ranging from about 0.001% to about 10%.

27. (canceled)

28. A method for treating inflammation associated with gram-negative folliculitis in a subject, said method comprising topically administering a therapeutically effective amount of a formulation of besifloxacin at a concentration ranging from about 0.5% to about 4%.

29. The method of claim 28, wherein the gram-negative folliculitis is caused by gram-negative bacteria that is resistant to conventional antibiotics and fluoroquinolone other than besifloxacin.

30.-38. (canceled)
